SEQUENCE LISTING (25 sequences)

SEQ ID NO: 1 - protein, 9 aa, Artificial Sequence
  Synthetic tumor-homing peptide
CRGDKGPDC

SEQ ID NO: 2 - protein, 31 aa, Artificial Sequence
  Synthetic tumor-homing peptide
KDEPQRRSARLSAKPAPPKPEPKPKKAPAKK

SEQ ID NO: 3 - protein, 9 aa, Artificial Sequence
  Synthetic tumor-homing peptide
CGNKRTRGC

SEQ ID NO: 4 - protein, 7 aa, Artificial Sequence
  Synthetic tumor-homing peptide
HAIYPRH

SEQ ID NO: 5 - protein, 7 aa, Artificial Sequence
  Synthetic tumor-homing peptide
ATWLPPR

SEQ ID NO: 6 - protein, 6 aa, Artificial Sequence
  Synthetic tumor-homing peptide
RRKRRR

SEQ ID NO: 7 - protein, 13 aa, Artificial Sequence
  Synthetic tumor-homing peptide
EEEAAGRKRKKRT

SEQ ID NO: 8 - protein, 36 aa, Artificial Sequence
  Synthetic tumor-homing peptide
AEQNPIYWARXADWLFTTPLLLLDLAALLVDADEGT

SEQ ID NO: 9 - protein, 29 aa, Artificial Sequence
  Synthetic tumor-homing peptide
WEAALAEALAEALAXHLAALAEALEALAA

SEQ ID NO: 10 - protein, 7 aa, Artificial Sequence
  Synthetic tumor-homing peptide
APSPMIW

SEQ ID NO: 11 - protein, 7 aa, Artificial Sequence
  Synthetic tumor-homing peptide
LQNAPRS

SEQ ID NO: 12 - protein, 10 aa, Artificial Sequence
  Synthetic tumor-homing peptide
CGLIIQKNEC

SEQ ID NO: 13 - protein, 10 aa, Artificial Sequence
  Synthetic tumor-homing peptide
CNAGESSKNC

SEQ ID NO: 14 - protein, 5 aa, Artificial Sequence
  Synthetic tumor-homing peptide
CREKA

SEQ ID NO: 15 - protein, 5 aa, Artificial Sequence
  Synthetic tumor-homing peptide
CGKRK

SEQ ID NO: 16 - protein, 7 aa, Artificial Sequence
  Synthetic tumor-homing peptide
CGSLVRC

SEQ ID NO: 17 - protein, 9 aa, Artificial Sequence
  Synthetic tumor-homing peptide
CPGPEGAGC

SEQ ID NO: 18 - protein, 5 aa, Artificial Sequence
  Synthetic tumor-homing peptide
CDTRL

SEQ ID NO: 19 - protein, 6 aa, Artificial Sequence
  Synthetic tumor-homing peptide
RRPYIL

SEQ ID NO: 20 - protein, 12 aa, Artificial Sequence
  Synthetic tumor-homing peptide
EDYELMDLLAYL

SEQ ID NO: 21 - protein, 7 aa, Artificial Sequence
  Synthetic tumor-homing peptide
ATWLPPR

SEQ ID NO: 22 - protein, 13 aa, Artificial Sequence
  Synthetic tumor-homing peptide
CGGKLKSQLVKRK

SEQ ID NO: 23 - protein, 12 aa, Artificial Sequence
  Synthetic tumor-homing peptide
CGGKNGRYSISR

SEQ ID NO: 24 - protein, 16 aa, Artificial Sequence
  Synthetic tumor-homing peptide
CGGRDGTRYVQKGEYR

SEQ ID NO: 25 - protein, 14 aa, Artificial Sequence
  Synthetic tumor-homing peptide
CGGAHWQFNALTVR

(19) United States
(12) Patent Application Publication
(10) Pub. No.: US 2013/0243741 A1
(43) Pub. Date: Sep. 19, 2013

(54) RENILLA/GAUSSIA TRANSFECTED CELLS AS A LIGHT SOURCE FOR IN-SITU PHOTODYNAMIC THERAPY OF CANCER

(71) Applicants: Stefan H. Bossmann, Manhattan, KS (US); Deryl L. Troyer, Manhattan, KS (US); Matthew T. Basel, Manhattan, KS (US); Tej B. Shrestha, Manhattan, KS (US); Hongwang Wang, Manhattan, KS (US)

(72) Inventors: Stefan H. Bossmann, Manhattan, KS (US); Deryl L. Troyer, Manhattan, KS (US); Matthew T. Basel, Manhattan, KS (US); Tej B. Shrestha, Manhattan, KS (US); Hongwang Wang, Manhattan, KS (US)

(73) Assignee: KANSAS STATE UNIVERSITY RESEARCH FOUNDATION, Manhattan, KS (US)

(21) Appl. No.: 13/821,516

(22) PCT Filed: Sep. 19, 2011
(86) PCT No.: PCT/US11/52156

§ 371 Date: May 9, 2013

Related U.S. Application Data
(60) Provisional application No. 61/384,957, filed on Sep. 21, 2010.

(51) Int. Cl. A61K 41/00

(52) U.S. Cl. CPC A61K 41/0057
USPC 424/93.21

(57) ABSTRACT

A method for photodynamic therapy treatment of cancerous cells and tissue is provided. The method comprises administering tumor-trophic cells expressing a luminescent protein to a subject. A photosensitizing agent is then separately administered to the subject, followed by an optional iron chelator. On the day of treatment, a luminogenic substrate corresponding to the luminescent protein is administered to the subject. The substrate reacts with the luminescent protein in the vicinity of the cancerous tissue to produce light which activates the photosensitizing agent resulting in the selective destruction of the cancerous tissue.

### CROSS-REFERENCE TO RELATED APPLICATIONS

The present application claims the priority benefit of U.S. Provisional Patent Application Ser. No. 61/384,957, filed Sep. 21, 2010, entitled Renilla/Gaussia Transfected Cells as Light Source for In-Situ Photodynamic Therapy of Cancer, incorporated by reference in its entirety herein.

### FEDERALLY SPONSORED RESEARCH OR DEVELOPMENT

This invention was made with government support under Grant No. 933701, awarded by the National Science Foundation. The United States government has certain rights in the invention.

### SEQUENCE LISTING

The following application contains a sequence listing in computer readable format (CRF), submitted as a text file in ASCII format entitled “SequenceListing,” created on Sep. 13, 2011, as 7 KB. The content of the CRF is hereby incorporated by reference.

### BACKGROUND OF THE INVENTION

1. Field of the Invention

The present invention relates to improved methods of photodynamic therapy (PDT) and imaging for deep tissue tumors.

2. Description of Related Art

PDT is used to treat a variety of medical conditions, including cancers. Broadly, PDT involves irradiation of a photosensitizer (used interchangeably herein with photosensitizing agent) with a light at a specified wavelength to induce oxidative damage in target tissues, such as tumors or other cancerous tissues. Photosensitizers generally have little or no toxicity in the absence of light. However, when most photosensitizers are illuminated by light of the correct wavelength and power, they absorb energy that they, in turn, transfer to oxygen present in the tissue, which ordinarily exists in the triplet electronic state. This energy transfer converts triplet oxygen to singlet oxygen, an extremely reactive species that is destructive to cells. PDT is considered minimally invasive and minimally toxic. Collateral tissue damage is usually minimized because the photosensitizer has either been selectively taken up by the target tissue and/or accumulates in the target tissue while being cleared quickly from surrounding, healthy tissue at the time of treatment. Damage to other tissue can also be minimized because the tissues that are meant to be destroyed are selectively illuminated and the damage is thus confined only to those areas. Mitochondria seem to be central coordinators of the mechanisms by which PDT induces apoptosis. Recent studies indicate that concomitant to the permeabilization of the outer mitochondrial membrane (which leads to the release of several apoptogenic factors in the cytosol and to the activation of effector caspases), regulatory signaling pathways are activated, depending on the chemical nature of the photosensitizer, the incident light energy, and the nature of the treated cancer cells. Thus, in addition to apoptosis, PDT also works to knock-out the energy producing mitochondria of cells, as well as stimulate the patient's own immune system to work against the target tissue.

Despite these advantages, PDT's uses are severely limited due to the low penetration depth of light into tissue, with many light wavelengths only being able to penetrate ⅓-inch of skin or less.

In particular, based upon the light absorption coefficients of water and human Aorta tissue (see FIG. 1), the minimum region of tissue absorption is at 800±50 nm. This corresponds to a light penetration depth (δ) of approximately 1 cm at 800 nm, 0.5 cm at 600 nm and 900 nm, and 0.1 cm at 400 nm.

Thus, PDT is generally limited to surface cancers, such as skin melanomas. Numerous strategies for enhanced irradiation of tumors that are located within the human or mammalian body are discussed in the literature. Among these approaches is the use of high-energy lasers (instead of lamps), laser diodes, the use of gold nanoshells and nanocages as high absorption and scattering materials, and bi- and multi-photon excitation of suitable chromophores. Although laser light sources are regarded as an improvement when compared to lamps, their very narrow bandwidth excitation requires the use of an appropriate laser-source for virtually each photosensitizer. Bi- and multiphoton absorption offers the advantage of spatially resolved irradiation and higher selectivity than mono-photonic excitation, especially when femtosecond pulses are used. However, the light intensities required for the simultaneous absorption of two or several photons are very hard to achieve when treating tumors within the human/mammalian body, because the two- and three-photon absorption cross-sections are too low. For deeper tissues, such as in lung or esophageal cancer, one of the current methods involves the use of a fiber optic cable inserted through an endoscope into the body to irradiate the target tissue. However, this results in a treatment protocol that is much more invasive than traditional PDT. Other efforts have been made to develop photosensitizers capable of working at higher wavelengths, which can penetrate deeper into the skin. However, such methods also have drawbacks. In particular, the deeper the penetration of the light, the less focused the light becomes and the more nonspecific the treatment becomes as an increasing amount of surrounding non-target tissue also gets irradiated and damaged during the process.

Thus, despite advances, PDT is still considered a treatment option primarily suitable for surface tissues and localized cancers, and not for use with deep-tissue tumors or cancers that have metastasized. Although PDT has been successful in treating tumors conveniently located to permit high doses of incident light, there remains a need in the art for PDT treatments applicable to all types of cancers, including deep-tissue tumors.

### SUMMARY OF THE INVENTION

The present invention is broadly concerned with new methods for using photodynamic therapy to treat, destroy, and kill cancerous tissue. In one or more embodiments, the invention provides a method for photodynamic therapy of cancerous tissue. The method comprises administering to a subject a therapeutically effective amount of tumor-trophic cells comprising a nucleic acid encoding for a luminescent protein. A photosensitizing agent is also administered to the subject separately from the cells. An iron chelator can optionally be administered to the subject, and then a luminogenic substrate corresponding to the luminescent protein is administered to the subject. Advantageously, the substrate reacts with the luminescent protein to produce light in situ (intracellularly), and the light activates the photosensitizing agent, which results in the damage and destruction of the cancerous tissue according to the various mechanisms for cell death described herein.

In one or more embodiments, there is also provided a method of imaging cancerous tissue. The method comprises administering to a subject tumor-trophic cells, which comprise a nucleic acid encoding for a luminescent protein. A photosensitizing agent is also administered to the subject separately from the cells. Advantageously, the cells and photosensitizing agent accumulate in and near the cancerous tissue. An iron chelator can optionally be administered to the subject, and then a luminogenic substrate corresponding to the luminescent protein is administered to the subject. The substrate reacts with the luminescent protein to produce light of a first wavelength. This light (energy) activates the photosensitizing agent, which emits light of a second wavelength. The method further comprises detecting this light of a second wavelength emitted from the photosensitizing agent to determine the location of the cancerous tissue in the subject.

A kit for the photodynamic therapy or imaging of cancerous tissue is also provided by the present invention. The kit comprises a photosensitizing agent comprising a tumor-homing peptide attached thereto; an optional iron chelator; a luminogenic substrate; and instructions for the administration thereof. The kit can also include instructions for selecting and transfecting tumor-trophic cells to deliver a luminescent protein to the cancerous tissue as the light source for the photodynamic therapy or imaging. Alternatively, the kit itself can include a suitable plasmid or vector comprising a nucleic acid encoding a luminescent protein, along with and instructions for transfecting a cell to deliver the luminescent protein to cancerous tissue as the light source for the photodynamic therapy or imaging. Similarly, the kit could also include the transfected cells themselves for use in the photodynamic therapy or imaging.

In one or more embodiments a further method for photodynamic therapy of cancerous tissue is provided. The method comprises (a) administering to a subject a therapeutically effective amount of tumor-trophic cells comprising a nucleic acid encoding for a luminescent protein; (b) administering a photosensitizing agent to the subject, wherein the photosensitizing agent is administered separately from the cells; (c) optionally administering a iron chelator to the subject; (d) administering a luminogenic substrate corresponding to the luminescent protein to the subject; and (e) repeating steps (a)-(d). Advantageously, the substrate reacts with the luminescent protein to produce light in situ (intracellularly), and the light activates the photosensitizing agent, which results in the damage and destruction of the cancerous tissue according to the various mechanisms for cell death described herein. By repeating the process more and more of the tissue is destroyed until the subject is preferably cancer-free. Repetition also helps to further stimulate the subject's own immune response.

### BRIEF DESCRIPTION OF THE DRAWINGS

FIG. 1 is a graph of the absorption coefficients of water and a typical human tissue (aorta) as a function of wavelength, drawn using data from the Handbook on Industrial Laser Safety;

FIG. 2 shows the normalized absorption and emission spectra of Renilla bioluminescence (light gray), RuC₂ light absorption (mid-gray), ³MLCTemission (mid-gray), TCPP light absorption (black), and fluorescence (black);

FIG. 3 shows the luminescence spectra of TCPP (light gray), TCPC (“chlorin;” black) and TCPBC (“BCl;” mid-gray);

FIG. 4 is a graph showing the concentration dependence of the LED/PDT-experiment in Example 2 at high incident power (P=0.0052 W);

FIG. 5 is a chart comparing the concentration dependence of the LED/PDT-experiments from Example 2 at low and high incident power;

FIG. 6a is an image of dye-loaded IFN-expressing hUCMS cells (gray) detected in small metastatic breast tumor (light gray), but not in the surrounding normal lung tissue (dark);

FIG. 6b is an image of Prussian blue stained NSCs (black) near B16F10 mouse melanoma cells (gray) 2 days after i.v. transplant;

FIG. 7a is a graph of the effect on B16FI0 melanoma cell viability of control cells (no treatment), ALA alone, coelenterazine alone, coelenterazine and NSC/GLuc, or full treatment (NSC/GLuc, ALA, coelenterazine) 12 hours after treatment in Example 3;

FIG. 7b shows the results from Example 3, 24 hours after treatment;

FIG. 7c shows the results from Example 3, 36 hours after treatment;

FIG. 8 is a graph of the percentages of dark areas in the lung of C57/BL6 mice bearing B16F10 lung melanomas in Example 4;

FIG. 9 is a graph of the results of the Mouse Survival Study after PDT of B16F 10luc2 Lung Tumors in C57BL6 mice from Example 5;

FIG. 10 shows IVIS images of a live mouse (Group 4) from Example 5 at day 25: (1) Directly after the injection of coelenterazine; (2) luminescence after 5 min.; (3) after 11 min.; (4) after 17 min.; (5) after 23 min.; and (6) after 28 min.;

FIG. 11 shows photographs of surgical confirmation of the tumor sites after euthanasia in the same mouse imaged in FIG. 10; V: ventral side, D: dorsal side;

FIG. 12 shows IVIS images of 4T1 breast cancer tumors in BALB C mice from Example 6;

FIG. 13 is a graph of the effect of neural stem cells on 4T1 tumor growth in Groups 1 and 2 from Example 6;

FIG. 14 is a graph comparing the results of Groups 2 and 3 from Example 6;

FIG. 15 is a graph showing the effect of the iron chelator, DFO, on tumor growth from Example 6; and

FIG. 16 is a graph comparing the results of all three test groups from Example 6.

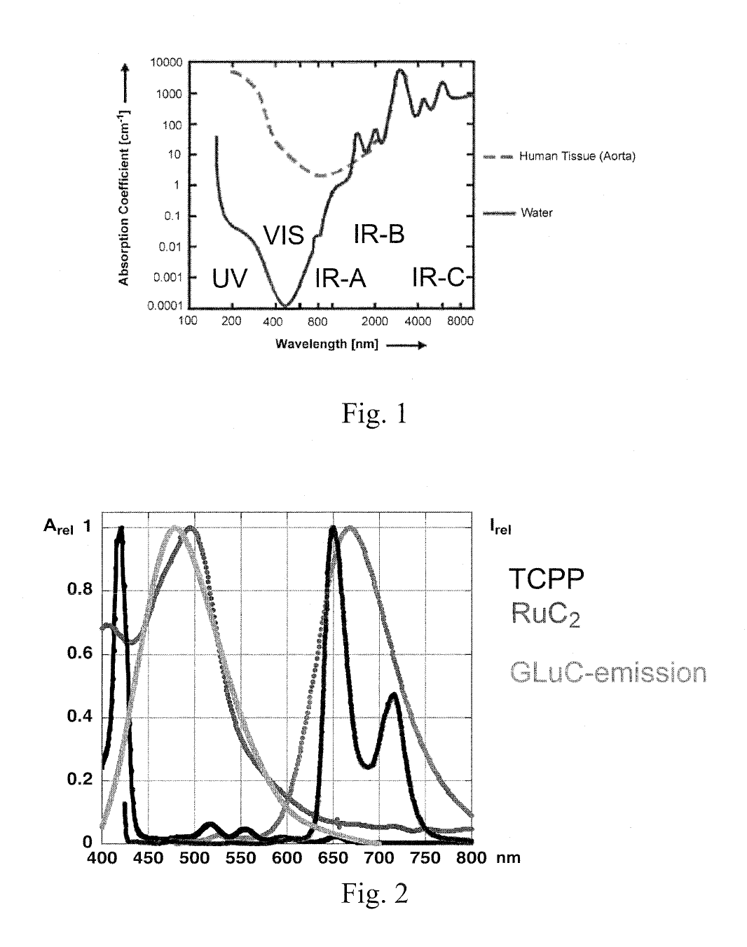

### DETAILED DESCRIPTION

In more detail, the present invention is concerned with new methods for photodynamic therapy of cancerous tissues using in-situ generation of light (a.k.a., intracellular generation of light) to activate the photosensitizing agent. More specifically, the present invention is concerned with luminescent cells as light sources for PDT, as well as various photosensitizing agents for use in the invention, and methods for PDT treatment of cancerous tissues.

Luminescent cells for use in the invention are preferably tumor-trophic cells with a tendency to migrate to cancerous tissues (i.e., the cells have an affinity for cancerous tissues, as opposed to normal or healthy tissue). Preferably, this affinity is inherent in the type of cell used; however, it will be appreciated that cells can also be modified for selective uptake by cancerous tissues using known methods. Particularly preferred cells for delivering the PDT light source to the cancerous tissue include mammalian stem cells (e.g., neural stem cells (NSCs), umbilical cord stem cells, mesenchymal stem cells, endothelial precursor cells, or induced pluripotent stem cells (iPSCs)), monocytes, and/or neutrophils. Tumors behave much like wounds that do not heal. Bone marrow mesenchymal stem cells and neural stem cells are known to migrate toward wounds and other areas of pathology and have been shown to be effective gene-delivery vehicles for targeted cancer therapy. Tumors secrete factors that recruit cells from surrounding tissue as well as from the bone marrow to provide support and nutrition. It has been shown that SDF1 alpha, EGF and PDGF, but not basic FGF or VEGF, enhance bone marrow MSC migration to tumor cells. Breast cancer cells have also been reported to make the chemokines CCL5 and CCL2, which may be responsible for stem cells trafficking to them. Stem cells from Wharton's jelly of the umbilical cord, termed umbilical cord matrix stem cells (UCMSC), are multi-potent and have characteristics very similar to bone marrow MSCs. Neural stem cells are particularly well-suited for used in the invention, as they have been found to exhibit high affinity for cancerous tissue.

Regardless of the cell type, the cells used in the invention are transfected with a nucleic acid encoding for a luminescent protein, and more particularly for an amino acid sequence of a luminescent protein. The term transfection, as used herein, generally means the introduction of foreign substances (usually nucleic acids) into cells, and in this case, the introduction of substances imparting luminescence to the cells. The terms luminescent and luminescence, as used herein, refer to bioluminescence, with it being understood that bioluminescence is a type of chemiluminescence occurring in a living organism. Thus, the cells comprise any suitable (exogenous) nucleic acid (e.g., DNA, cDNA, mRNA, etc.) encoding a luminescent protein such that the transfected cells express a luminescent protein, and more preferably secrete a protein, which in the presence of the appropriate substrate, results in the emission of light in or near the cancerous tissue. Nucleic acids, optionally along with appropriate regulatory sequences (e.g., promoters, enhancers, etc.) can be introduced into the cells using lipids, plasmids, vectors, adenoviruses, lentiviruses, and the like. In one or more embodiments, the cells comprise a plasmid or vector comprising a nucleic acid (optionally, operatively linked to a suitable promoter and/or enhancer) encoding a luminescent protein. Any suitable transfection method can be used, including those using cationic liposomes, polyethylenimine (PEI), and/or electorporation. Transfection can be stable or transient. For transient transfection, the cells are preferably grown on a suitable growth medium (e.g., DMEM, RPMI, and/or Ham's F10), optionally with serum (e.g., FBS and/or horse). The exogenous nucleic acid to be transfected into the cells is diluted in a suitable growth medium. A suitable transfection reagent (e.g., TurboFect™, Lipofectamine™, and/or PEI) is added to the nucleic acid and mixed. Transfection reagents will neutralize or even create an overall positive charge on the negatively charged nucleic acid molecule, allowing it to cross the negatively charged cell membrane. The transfection reagent/DNA mixture is then combined with the cells. The cells can then be incubated in a CO₂ incubator at about 37° C. for about 24-48 hours to yield the transfected cells. It will be appreciated that the amount of DNA and transfection reagent used will depend upon the number of cells to be transfected, as well as the cell type.

A preferred luminescent protein for use in the invention is the oxidative enzyme, luciferase. Thus, in one or more embodiments, the luminescent cells according to the invention preferably comprise a luciferase gene encoding for the luminescent protein. Luminescent proteins are preferably selected from the group consisting of Renilla luciferase, Gaussia luciferase, firefly luciferase and combinations thereof, with Gaussia luciferase being particularly preferred. Renilla luciferase is a 36 kDa protein from the sea pansy (Renilla renifirmis). Gaussia luciferase is a 19.9 kDA protein from the calanoid copepod (Gaussia princeps), a member of the phylum Arthropoda. Compared to the widely used Firefly luciferase, which is a 61 kDa protein isolated from a beetle (Photinus pyralis), Renilla luciferase is generally considered inferior when used for bioimaging, because its bioluminescence maximum is blue-shifted by approximately 70 nm (480 nm instead of 550 nm). However, unlike Firefly luciferase, which requires luciferin in the presence of oxygen, ATP, and magnesium to produce light, Renilla luciferase and Gaussia luciferase require only oxygen and the luminogenic substrate coelenterazine to produce light. ATP is not required. Both Renilla luciferase and Gaussia luciferase catalyze the oxidation of coelenterazine to yield coelenteramide and blue light of about 480 nm (λmax). Advantageously, Gaussia luciferase has also been found to be 750-fold brighter than native Renilla luciferase (when consuming the same amount of coelenterazine).

Any suitable luciferase nucleic acid or amino acid sequence can be used in the invention, including native forms, modified forms, synthesized forms, mutant forms, recombinant forms, and/or clones, etc. Luciferase genes, as well as plasmids and vectors containing luciferase genes are commercially-available and known in the art. For example, various Gaussia and Renilla luciferase sequences and proteins are described in U.S. Pat. Nos. 7,939,649, 7,507,565, 7,524,674, 6,232,107, 6,436,682, 5,292,658, and 5,418,155, incorporated by reference herein in their entireties to the extent not inconsistent with the present disclosure. Expression vectors and plasmids for Gaussia luciferase are commercially available from Targeting Systems (El Cajon, Calif.), including pCMV-GLuc, pGLuc-Basic, pBasic-GLuc-KDEL, pCMV-GLuc-KDEL, and plenti-GLuc-EGFP, or from Nanolight Technology (Pinetop, Ariz.), including pUC18 GLuc, pUC18 SS-GLuc, pCMV-Gluc-1, pGluc-basic-1, pCMV-KDEL-Gluc-1, pGLuc-KDEL-Basic-1, and pCMV-Gluc minus SS. Renilla luciferase expression vectors and plasmids are also available from Nanolight, including pUC19 R.M. Luc, as well as Promega (Madison, Wis.), including pRL-SV40, pRL-TK, pRL-CMV, and pRL-null. Plasmids containing Renilla luciferase variants including RLuc8, RLuc/M185V/Q235A, RLuc8.6-535, and Rluc7-525 are also available from Stanford University.

In use, a therapeutically-effective amount of transfected cells is administered to a subject. As used herein, a “therapeutically effective” amount refers to the amount of the cells or other component of the treatment method that will elicit the biological or medical response of a tissue, system, animal, or human that is being sought by a researcher or clinician, and in particular elicit some desired therapeutic effect. For example, in one or more embodiments, a therapeutically effective amount of the luminescent cells is an amount that produces sufficient light to damage or destroy (via apoptosis) the target tissue (e.g., cancer cells). One of skill in the art recognizes that an amount maybe considered therapeutically effective even if the condition is not totally eradicated but improved partially. The luminescent cells can be injected directly into the target tissue, or can be administered systemically. Advantageously, even when administered systemically, the luminescent cells preferentially accumulate in the cancerous tissue, and preferably actively integrate in the cancerous tissue, as opposed to surrounding healthy tissue. This preferably occurs within about 2 to about 5 days after administration, depending upon the subject (with about 3 to about 5 days being the preferred time period for human subjects). More specifically, the cells can be administered using any suitable method including intravenous (i.v.), intraperitoneal (i.p.), intramuscular (i.m.), intratumoral (i.t.), intraarterial (i.a.), and/or inhalation. Intravenous administration is particularly preferred for solid tumors, while i.p. administration is preferred for pancreatic, liver, and gastric tumors. Inhalation is also a particularly suitable administration method for lung cancers. In one or more embodiments, the cells are first dispersed in a pharmaceutically-acceptable carrier or excipient before be administered to the subject. As used herein, the term “pharmaceutically-acceptable” means not biologically or otherwise undesirable, in that it can be administered to a subject without excessive toxicity, irritation, or allergic response, and does not cause any undesirable biological effects or interact in a deleterious manner with any of the other components of the composition in which it is contained. A pharmaceutically-acceptable carrier or excipient would naturally be selected to minimize any degradation of the active ingredient and to minimize any adverse side effects in the subject, as would be well known to one of skill in the art. Pharmaceutically-acceptable ingredients include those acceptable for veterinary use as well as human pharmaceutical use. Exemplary carriers and excipients include aqueous solutions such as normal (n.) saline (−0.9% NaCl), phosphate buffered saline (PBS), sterile water/distilled autoclaved water (DAW), as well as cell growth medium (without serum, e.g., DMEM), aqueous solutions of dimethyl sulfoxide (DMSO), polyethylene glycol (PEG), and/or dextran (less than 6% per by weight.)

The amount of cells administered per each injection/dose will depend upon the subject, but can range from about 500,000 to about 200 million cells, with up to about 100 million cells preferably being administered per injection/dose for human therapies.

A photosensitizing agent is then administered to the subject, preferably within about 1 to about 7 days after administering the cells (more preferably about 2 to about 5 days). The agent can be administered locally or systemically using any suitable administration method described herein, with i.v. being particularly preferred. The agent can first be dissolved or dispersed in any pharmaceutically-acceptable carrier or excipient described herein, with a preferred carrier being n. saline, or an aqueous solution of DMSO, PEG, and/or dextran. The photosensitizing agent is selected so that the absorption spectrum of the photosensitizer is tuned to the emission spectrum of the luminescent cells, so that it can be activated by the bioluminescence generated from the cells.

Thus, it will be appreciated that the particular photosensitizing agent(s) used will depend upon the luminescent protein selected. Although the mechanism may vary, once activated, the photosensitizing agents will preferably result in apoptosis, as opposed to necrosis, of the target tissue. The selection of the photosensitizing agent will also depend upon whether imaging of the cancerous tissue is also desired, as discussed in more detail below. Exemplary photosensitizing agents include free or metalated (e.g., Zn, Al, Ru) porphyrins, chlorins, benzoporphyrins, phthalocyanins, bacteriochlorins, cyanines, ruthenium complexes (especially ruthenium(II)-polypyridyl complexes), photosensitizer-generating prodrugs (e.g., 5-aminolevulinic acid (“ALA”)), and mixtures or combinations thereof. Combinations of one or more photosensitizing agents can be used. The agents can be administered as part of the same composition (i.e., one injection), or can be administered as part of different composition (i.e., multiple injections, with each injection administering a different photosensitizing agent).

For porphyrins, upon light absorption by the Q-band (λ=480 to 675 nm), which corresponds to the ¹ΠΠ*-transition, or Soret band, being the ²ΠΠ*-transition, a reactive singlet state is generated. It then undergoes efficient intersystem crossing to its much longer-lived triplet state. A typical

(³ΠΠ*) triplet state exhibits a lifetime in the microsecond to the millisecond range. During that time, dissolved oxygen molecules (³O₂) have to diffuse within the energy-transfer range of the triplet state (approx. 10-30 nm) to permit energy-transfer from ³ΠΠ* to ³O₂, resulting in the formation of singlet oxygen (¹O₂) and the electronic ground state of the sensitizer. Singlet oxygen is a very reactive species, which can damage biomolecules including proteins, nucleic acids, carbohydrates and (membrane) lipids via addition to double bonds or electron transfer reactions. The latter are known to initiate Advanced Oxidation Processes (AOP's) involving a manifold of highly reactive intermediates, such as the superoxide anion (O₂^{.-}), hydroperoxyl-radicals (R—O—O) and the hydroxyl radical (HO.). However, all of those processes consume dissolved oxygen (³O₂) and can therefore be severely hampered by hypoxia.

During the last two decades, ruthenium(II)-polypyridyl complexes have been extensively used to elucidate the structure and the biophysical properties of various types of DNA. All of these Ru(II)-complexes possess emissive ³MLCT (metal to ligand charge transfer) states and facilitate, to various degrees, photo-electron transfer reactions and/or the generation of singlet oxygen. Although these studies have uncovered many very interesting mechanistic insights, they were of very little importance with respect to photodynamic therapy because ruthenium(II)polpyridyls do not absorb significantly beyond 600 nm. However, the use of an in-situ light source, which matches their spectral characteristics, such as the luciferase transfected cells described herein, opens a new class of PDT-agents with improved photophysical qualities. Particularly preferred photosensitizing agents include tetrakis-4-carboxyphenyl-porphyrin (TCPP), Tris(N,N′″-bis(2-carboxyethyl)-4,4′:2,2″:4″,4′″-Quaterpyridine-N′N″-dium-N′,N′″) ruthenium(II)octachloride (RuC₂), tetra-carboxyphenyl-chlorin sodium salt (TCPC), 5 tetra-carboxyphenyl-bacteriochlorin sodium salt (TCPBC), meso-tetra-hydroxophenyl-chlorin (mTHPC), aminolevulinic acid (ALA), and/or PHOTOFRIN® (porfimer sodium).

RuC₂ is especially suited for PDT as it posses a very high oxidation potential E₀ Ru^{3+/2}=1.537 V (vs. SHE) at pH=7.0. Its long-lived ³MLCT-state (several hundred nanoseconds in aerated aqueous solutions) and its very high photochemical reduction potential E₀*Ru^{2+/1+}=1.670 V also make it a very damaging agent of DNA and other vital cell functions. The Ru²⁺ state is able to reduce O₂ to O₂⁻ (superoxide anion), another potent cell toxin (E₀ Ru^{2+/1+}=−0.477 V).

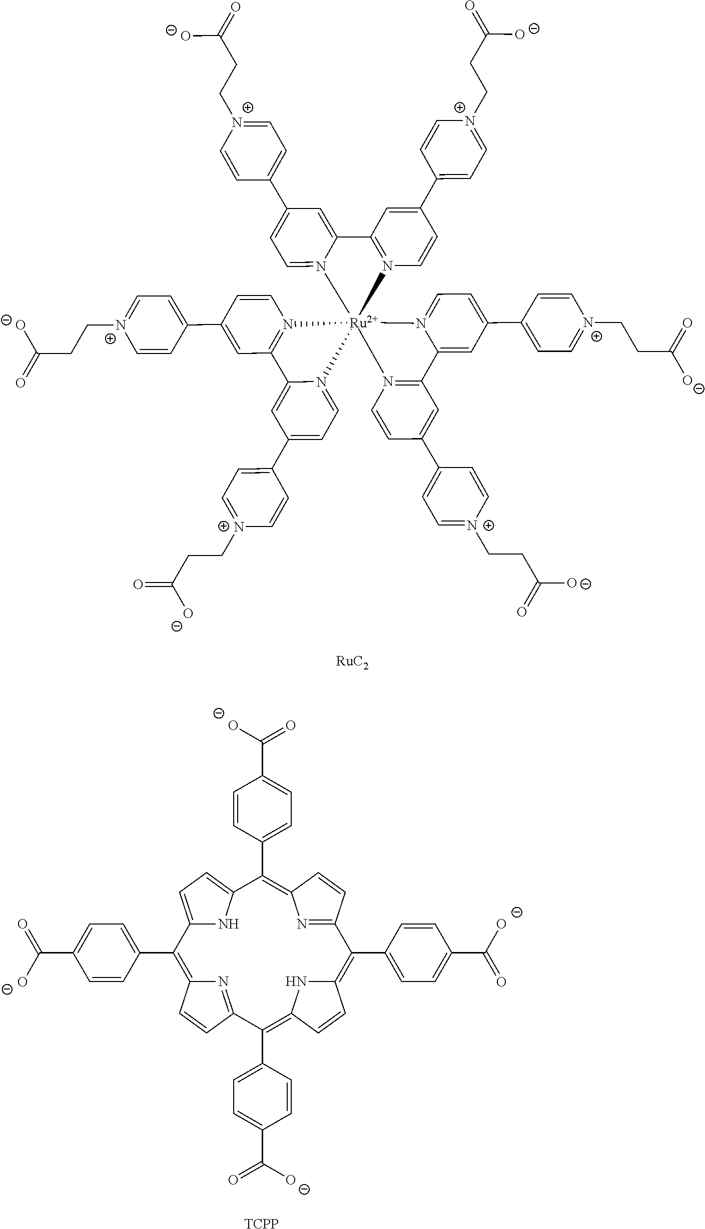

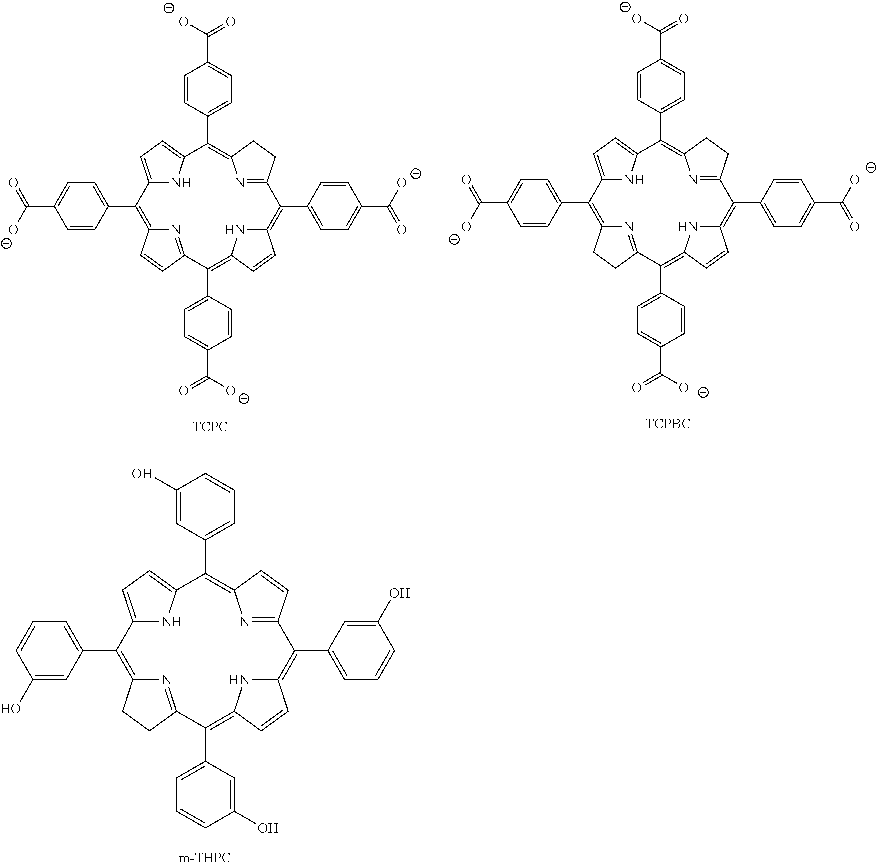

Prodrugs that result in the formation of photosensitizing agents in vivo can also be used. One example is ALA (mentioned above), a precursor for protoporphyrin IX.

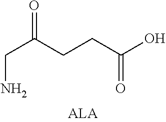

ALA is the substrate for the biosynthesis of protoporphyrin IX in the mitochondria of the cancer cells. Administration of ALA results in the formation of protoporphyrin IX in vivo, which acts as the photosensitizer in the PDT treatment process.

In one or more embodiments, a tumor-homing peptide sequence can be attached to the photosensitizing agent to direct the agent to, and cause it to accumulate in, the cancerous tissue, even when administered systemically. That is, when the tumor-homing peptides are used, direct injection into the cancerous tissue is not necessary. The additional advantage to systemic administration of the PDT components is that cancerous tissue that has not yet even been identified can benefit from this treatment. Examples of suitable tumor-homing sequences, which can be coupled using any suitable method (e.g., amide, CDI, etc.) are listed in the table below.

TABLE
Tumor-homing peptide sequences

| Name or | | |
|---|---|---|
| Acronym | Sequence | Binding site |
| iRGD | CRGDKGPDC (SEQ ID NO. 1) | Tumor endothel. |
| F3 | KDEPQRRSARLSAKPAPPKPEPKPKKAPAKK | Nucleolin, endothel., cells |
| | (SEQ ID NO. 2) | |
| LyP-1 | CGNKRTRGC (SEQ ID NO. 3) | Tumor lymphatic |
| TFR targeting | HAIYPRH (SEQ ID NO. 4) | Transferrin receptor |
| | | (non transferring binding part) |
| KDR binder | ATWLPPR (SEQ ID NO. 5) | Angiogenic vessels |
| VEGFR binder | RRKRRR (SEQ ID NO. 6) | Angiogenic vessels |
| Glu-Oct6 | EEEAAGRKRKKRT (SEQ ID NO. 7) | Glutamate receptor e.g., psma |
| pHLIP | AEQ NPIYWARXADWL | pH dependant |
| | FTTPLLLLDLAALLVDADEGT, | |
| | where X = 3,5 diiodotyrosine | |
| | (SEQ ID NO. 8) | |
| YALA | WEAALAEALAEALAXHLAALAEALEALAA, | pH sensitive; delivers across |
| | where X = 3,5 diiodotyrosine | cell at pH 6.7 |
| | (SEQ ID NO. 9) | |
| CD133 binding | APSPMIW (SEQ ID NO. 10) | |
| CD133 binding | LQNAPRS (SEQ ID NO. 11) | |
| CLT 1 | CGLIIQKNEC (SEQ ID NO. 12) | Clots in tumors |
| CLT2 | CNAGESSKNC (SEQ ID NO. 13) | Clots in tumors |
| | CREKA (SEQ ID NO. 14) | Fibrin clots |
| | CGKRK (SEQ ID NO. 15) | Heparan sulfate |
| | CGSLVRC (SEQ ID NO. 16) | Tumor vasc. |
| | CPGPEGAGC (SEQ ID NO. 17) | Tumor vasc. |
| | CDTRL (SEQ ID NO. 18) | Tumor vasc. |
| | RRPYIL (SEQ ID NO. 19) | Neurotensin receptor |
| | EDYELMDLLAYL (SEQ ID NO. 20) | |
| | ATWLPPR (SEQ ID NO. 21) | VEGF receptor; Tumor vasc. |
| | CGGKLKSQLVKRK (SEQ ID NO. 22) | HA |
| | CGGKNGRYSISR (SEQ ID NO. 23) | HA |
| | CGGRDGTRYVQKGEYR (SEQ ID NO. 24) | HA |
| | CGGAHWQFNALTVR (SEQ ID NO. 25) | HA |

For example, RuC₂ with the tumor-homing peptide sequence iRGD attached via a classic amide coupling procedure is depicted below.

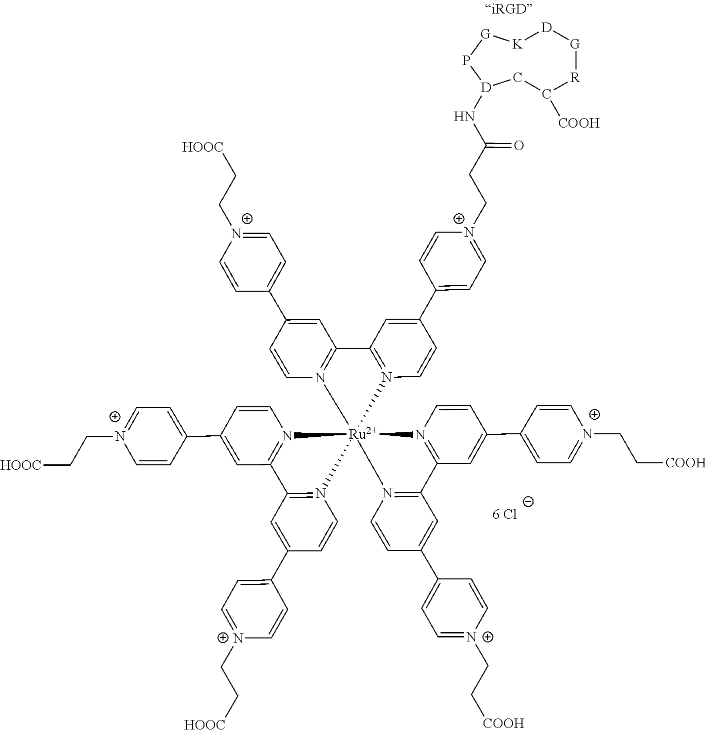

Likewise, m-THPC with iRGD coupled using a carbonylimidazole (CDI) procedure is depicted below.

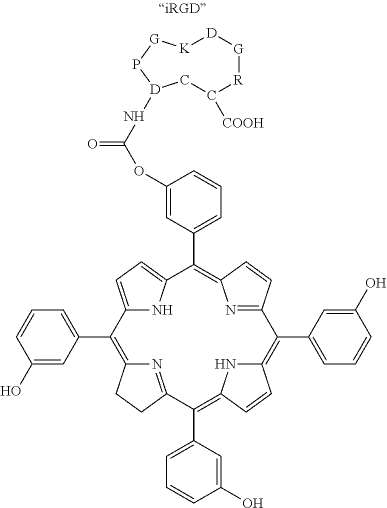

A tumor-homing peptide is not necessary for use with ALA.

The dosage amount for the photosensitizing agent will depend upon the agent used. For example, m-THPC (with iRGD) is preferably administered at a dosage level (for each administration) of from about 0.1 to about 5 mg/kg weight of the subject, and more preferably at a level of from about 0.3 to about 1 mg/kg body weight of the subject. Similarly, RuC₂ (with iRGD) is preferably administered at a dosage level (for each administration) of from about 0.1 to about 5 mg/kg weight of the subject, and more preferably from about 0.875 to about 2 mg/kg body weight of the subject. On the other hand, ALA is preferably administered at a dosage level (for each administration) of from about 10 to about 500 mg/kg weight of the subject, and more preferably about 100 mg/kg body weight of the subject.

Depending upon the photosensitizing agent used, an iron chelator can be administered to the subject about 1 day before PDT treatment. Suitable iron chelators include deferoxamine (DFO) and/or dipyridylthiosemicarbazone-chelators (e.g., DP44 mT, Dp4eT, Dp4 mT, DPT, or TS-BP44 mT).

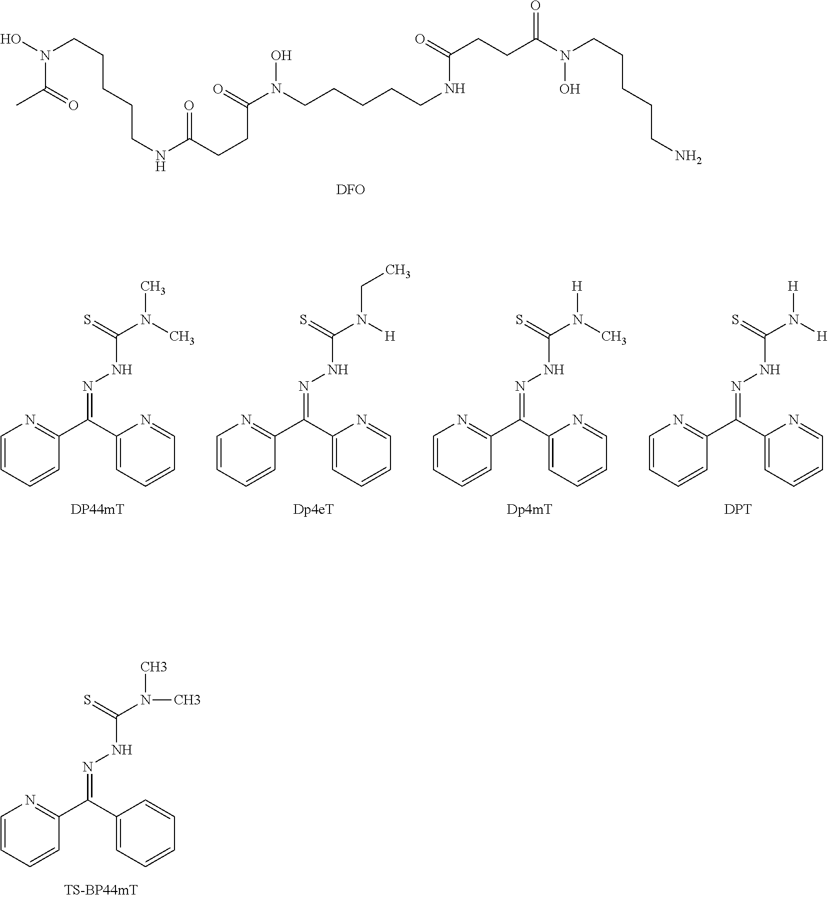

In particular, protoporphyrin IX is a direct precursor of heme, and specifically combines with iron to form heme. Thus, when the prodrug ALA is used for the photosensitizing agent, an iron chelator is also preferably used in the treatment method to maintain a sufficient amount of protoporphyrin free for PDT. The iron chelator can first be dissolved or dispersed in a pharmaceutically-acceptable carrier or excipient described herein, with a preferred carrier being an aqueous solution of DMSO, PEG, and/or dextran. When used, the iron chelator is preferably administered to the subject at a dosage level (for each administration) ranging from about 10 to about 2,000 mg/kg body weight of the subject depending upon the particular iron chelator used, with about 200 mg/kg being particularly preferred for DFO.

On the day of treatment, a luminogenic substrate is administered to the subject. The substrate can be administered via any suitable administration method described herein, with i.v. administration being preferred for human therapies. Retroorbital administration may be used for mice. The substrate can first be dispersed in any pharmaceutically-acceptable carrier or excipient described herein, with a preferred substrate being an aqueous solution containing beta-cyclodextrine (up to 2% by weight). Suitable substrates will depend upon the luminescent protein used in the cells. That is, the luminogenic substrate is selected so that it corresponds to the luminescent protein (i.e., so that the luminescent protein/enzyme will act upon the substrate molecule to produce the desired light reaction). Preferred substrates include coelenterazine and luciferin. As mentioned above, coelenterazine is the substrate used for Renilla and Gaussia luciferase. Coelenterazine can be used in its native form, although derivatives are preferred due to the low water solubility of coelenterazine. Several natural and synthetic derivatives are commercially-available, or can be synthesized according to known methods, as well as methods described herein. The cyclodextrin complex of coelenterazine is particularly preferred for use in the invention as imparting water-solubility to the coelenterazine molecule. A preferred method for synthesizing coelenterazine is also described herein.

The day of treatment is preferably from about 1 to about 7 days after administration of the luminescent cells (and preferably about 3 to about 5 days). The amount of substrate will depend on several factors; however, it is preferred that enough substrate is provided for at least about 10 minutes of light generation, more preferably at least about 30 minutes, and even more preferably from about 30 to about 60 minutes of light generation. For coelenterazine, the substrate is preferably administered at a dosage level (for each administration) in an amount ranging from about 5 to about 20 mg/kg weight of the subject. When administered as part of a complex, the coelenterazine complex will preferably be administered in sufficient amounts to provide coclenterazine at the above levels.

The PDT treatment process according to the invention operates according to the following mechanism (described with respect to a particularly preferred, but non-limiting, exemplary reaction). The luciferase-transfected cells which have accumulated in and around the cancerous tissue express and/or secrete the luminescent protein (enzyme). The luminescent protein, in the presence of the substrate (e.g., coelenterazine) and molecular oxygen, oxidizes the coelenterazine to produce light along with other by-products. This light, in turn, activates the photosensitizing agent, which has also accumulated in and around the cancerous tissue. The penetration depth of the light preferably ranges from about 0.1 cm at 480 nm to about 0.5 cm at 600 nm, and more preferably up to about 1 cm at 800 nm. The penetration depth is defined herein as the distance traveled (by the light) at which the incident light intensity has decreased to 1/e. Advantageously, due to the proximity of the photosensitizer and the light source to one another, a large penetration depth is not necessary to activate the system, even in deep tissue tumors. Thus, shorter wavelength light can be used to activate the photosensitizing agent. This increases the specificity of the inventive PDT treatment method and reduces damage to surrounding healthy tissue, which can be unnecessarily illuminated during other PDT treatment processes. Activation of the photosensitizer results in the permeabilization of the outer mitochondrial membrane (which leads to the release of several apoptogenic factors in the cytosol and to the activation of the effector caspases). Depending upon the chemical nature of the photosensitizer, the light energy, and the cells being treated, treatment also leads to regulatory signaling pathways being activated in the cancerous tissue, as discussed herein, which stimulate the immune response of the subject. In one or more embodiments, combinations of photosensitizing agents can be used. For example, it has been found that the use of TCPP and RuC₂ results in a synergistic effect that improves the treatment process.

The foregoing process can be repeated several times until all or substantially all of the cancerous tissue has been destroyed. That is, after the first treatment, luminescent cells can again be administered to the subject, followed by a photosensitizing agent, optional iron chelator, and substrate, as indicated above. Subsequent treatments can begin as soon as the preceding treatment is finished (i.e., after the substrate has been administered in the preceding treatment process). Repeating the treatment process ensures that all of the cancerous tissue is destroyed and increases the effect on the immune response against the cancerous tissue. For human therapies, treatment is preferably repeated at least about 5 times, and more preferably from about 10 to about 20 times, preferably over the course of about 30 to about 60 days.

Depending upon the photosensitizer used, this process can also be used to image the cancerous tissue. In particular, photosensitizers such as porphyrins and ruthenium complexes not only absorb the light emitted from the bioluminescent cells, but also emit their own light at a defined wavelength, which can be viewed using suitable imaging equipment. Thus, the luminescent cells can be used as an excitation light source similar to traditional imaging. However, this type of imaging has an advantage over fluorescence/phosphorescence-based imaging, which is dependent on incident light, because the background fluorescence is much lower and, therefore, bioluminescence-based imaging is up to 10 times more sensitive than fluorescence/phosphorescence tomography.

In one or more further embodiments, some or all of the components of the PDT treatment method, as described above, can be provided together in a kit to facilitate carrying out the treatment process.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

The invention described herein is discussed primarily with respect human-based cancer therapies; however, it will be appreciated that the treatment can be applied for clinical research or therapeutic treatment to any suitable animal, including, without limitation, dogs, cats, and other pets, as well as, rodents, primates, horses, cattle, etc. Additional advantages of the invention will be apparent to those in the art upon review of the disclosure herein and the working examples below

### EXAMPLES

The following examples set forth methods in accordance with the invention. It is to be understood, however, that these examples are provided by way of illustration and nothing therein should be taken as a limitation upon the overall scope of the invention.

### Example 1

### Initial Investigations

Using conventional transfection techniques according to the manufacturer's instructions, we have transfected C17.2. neural stem cells with a mammalian expression plasmid containing RLuc8 (Renilla luciferase pcDNA-RLuc8 plasmid, obtained from Dr. Loening; Stanford University, Stanford, Calif. 94305). The RLuc8 variant is approximately 4× brighter than native RLuc and about 200× more stable than RLuc in murine serum at 37° C. We hypothesized that the emitted light can be absorbed by RuC₂ and then (partially) transferred to TCPP, TCPC, and TCPBC, or directly absorbed by the porphyrin, chlorin, or bactcriochlorin.

As shown in FIG. 2, the emission spectrum of Renilla luciferase and the ³MLCT-absorption band of RuC₂ match perfectly. All measurements were taken at pH=6.8 in phosphate buffer using 4.0 mL quartz cuvettes (Helma) using a spectrofluorometer (Fluoromax 2) with dual monochromators and a diode array UV/Vis absorption spectrometer (HP 8453).

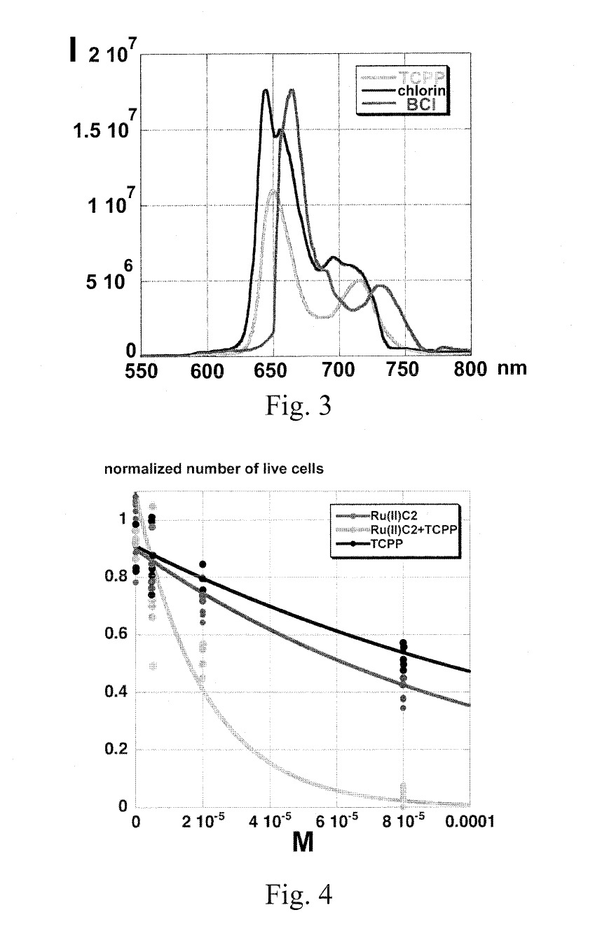

In FIG. 3, the luminescence spectra of TCPP, TCPC, and TCPBC are shown. The emission from the chlorin and the bacteriochlorin-derivative will be clearly discernible because of the distinct shifts compared to the porphyrin. Excitation wavelength: λ=480 nm. The absorption of all three samples at λ=480 nm was 0.10. All measurements were taken at pH=6.8 in phosphate buffer using 4.0 mL quartz cuvettes (Helma) using a spectrofluorometer (Fluoromax 2) with dual monochromators and a diode array UV/Vis absorption spectrometer (HP 8453). This emission can be used to track the luminescence occurring from all cancerous tissue thus permitting its simultaneous detection and a determination of cancer-related protease activity.

### Example 2

### Feasibility Study Using LED's

LED-irradiation experiments employing RL5-B12120 Superbright LED's, (Qmax=470 nm, light output 0.0114±0.0008 W) and a light-attenuation system consisting of a series of parallel optical filters was performed. After trypsinization, B16F10 mouse melanoma cells were counted using a hemacytometer and the trypan blue exclusion method, and plated in tissue culture plates (TPP brand, Midwest Scientific). Four hours post-plating, RuC₂, TCPP, or a 1:1 mixture of RuC₂ and TCPP was added to the plates, with the 1:1 mixture containing each agent at half their respective concentrations to permit comparisons at the same molar concentrations of PDT-agents. The cells were then stored in the dark for 24 hours. Next, the cells were irradiated with high or low intensity of LED light for 45 min. Twenty-four hours post-radiation, MTT cell proliferation reagent (thiazolyl blue) was added to the cells, which were then incubated four hours. Next, SDS-solubilization buffer was added and the cells were incubated for 12 hours. The plates were then read using a plate reader (Spectramax 190) at 550 nm and 690 nm (background establishment).

In FIG. 4, the concentration dependence of the LED/PDT-experiments at high incident power (P=0.0052 W) is shown. Strong synergy was observed when 4×10⁻⁵ M RuC₂ and 4×10⁻⁵ M TCPP were used together. It should be noted that in the absence of irradiation, the observed growth inhibition was approx. 20% for 8×10⁻⁵ M of RuC₂, and close to zero for TCPP.

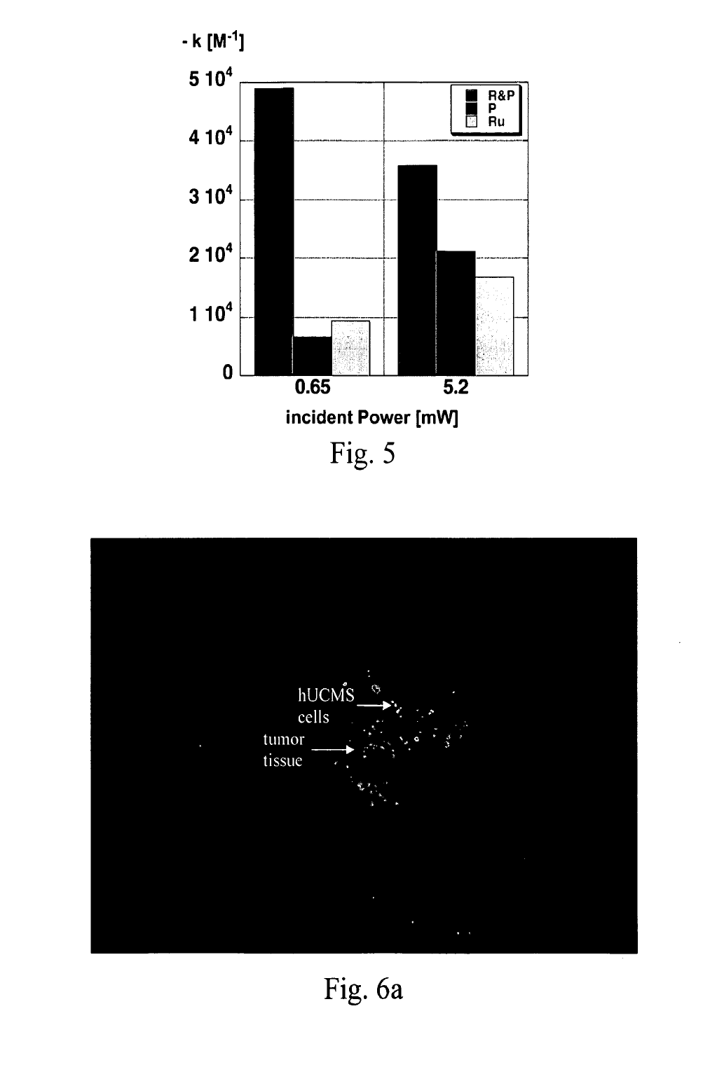

It was observed that the synergy between the two PDT-photosensitizing agents becomes even stronger under irradiation at low light intensity, as FIG. 5 indicates. It is estimated that the light intensity of 0.65 mW is the maximum of emission energy that can be expected from a bioluminescent irradiation system consisting of Renilla luciferase-transfected stem cells.

### Example 3

### In Vitro Study Using the Blue Luminescence from Gaussia Luciferase

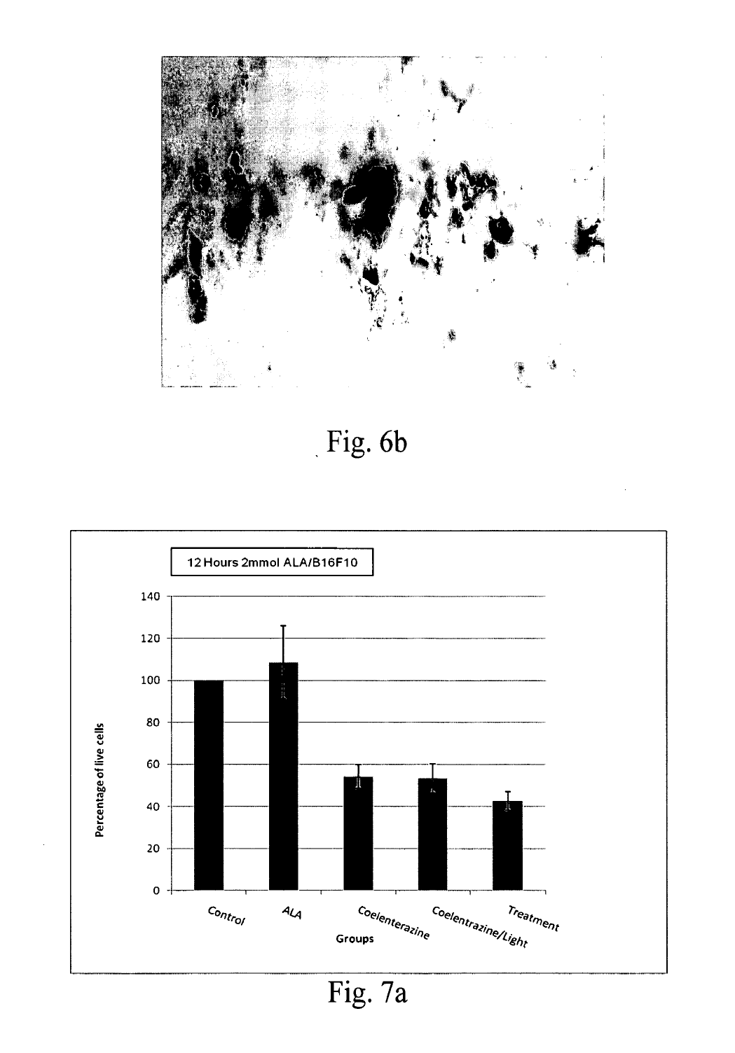

A co-culture system was used to study the photodynamic effect of Gaussia luciferase-expressing neural stem cells (NSCs) or rat umbilical cord matrix stem cells (rUCMSCs) on B16F10 melanoma model cells. In previous work, stem cells have been used as stealth vehicles in a more classical cell-based gene therapy approach using, cytokine-transfected hUCMSCs, which significantly attenuated experimental lung metastasis of breast cancer cells without evidence of any damage in other normal tissues. As shown in FIG. 6a, dye-loaded IFN-expressing hUCMS cells were detected in small metastatic breast tumor tissue (see arrows), but not in the surrounding normal lung tissue. The image was recorded using a Nikon Diaphot inverted microscope on a TMD airtable equipped with Hoffman illumination, epifluorescence, and Leica and Eppendorf microinjection system. Similarly, previous work has established that stem cells preferentially accumulate in cancerous tissue. For example, NSCs were found in the vicinity of B16F 10 mouse melanomas only 2 days after i.v. transplant. As shown in FIG. 6b, the Prussian blue stained NSCs (black) are visible near B16F10 mouse melanoma cells (gray). The image was recorded 2 days after transplant on a Leica DMRD microscope in combination with an Optronics 870 color CCD camera attached to a Bioquant True color image analysis system.

In the present Example, NSCs or rUCMSCs transfected with GLuc (2-5×10⁴ cells/well of each cell type) were suspended in 1.0 ml of the defined medium (i.e., DMEM). B16F10 mouse melanoma cells were cultured together with the transfected cells, and then incubated at 37° C. with 5% CO₂ for 24 hours for growth of colonies. The photosensitizing agent, aminolevulinic acid (2 mmol) was added when colonies became visible. Twenty-four hours later, cyclodextrin-complexed coelenterazine was added so that visible light was emitted from the NSCs or rUCMSCs. Various plates were set up as follows: control cells (no treatment), ALA alone, coelenterazine alone, coelenterazine and stem cell/GLuc, or full treatment (stem cell/GLuc, ALA, and coelenterazine).

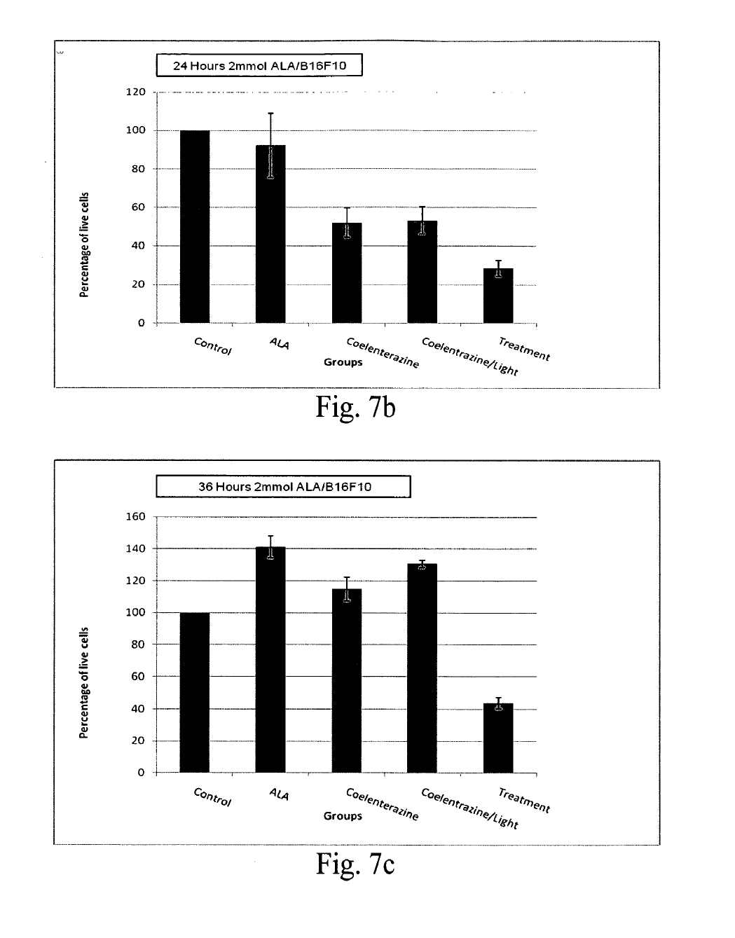

Colonies greater than 600 μm² were counted by an automated colony counter (Olympus CKX41 equipped with computer automated motor-drive stage and analysis system, St Louis, Mo.). After addition of the substrate, viable cancer cell numbers were analyzed for at 12-, 24-, and 36-hours using the MTT assay (Roche). All experiments were done in triplicate and repeated at least three times. The results are shown in FIG. 7. FIG. 7c shows that 36 hours after the beginning of the photodynamic treatment, the percentage of live cancer cells was significantly reduced in comparison to all other groups.

### Example 4

### In Vivo Study Using the Blue Luminescence from Gaussia Luciferase

This study was designed to determine whether photodynamic therapy for deeply located primary or metastatic neoplasia can be achieved by the generation of light from luciferase-expressing delivery cells that exhibit significant tumor-homing ability. This in vivo model tested a variety of phototoxic compounds with varying concentrations and combinations delivered by multifunctional nanoplatforms bearing ester-linked RuC₂ and TCPP.

Murine melanoma cells (B16/F 10), purchased from ATCC, were maintained on DMEM with 10% FBS. On Day 1 of the study, six-week old CB57BL/6 female mice (Charles River) were transplanted with 5×10⁵ B16/F10 melanoma cells intravenously (250,000 cells/mouse or more (200 μL volume)) to create a metastatic lung melanoma model. This was done under short-duration anesthesia via isoflurane. Respiration rate and depth were constantly monitored during the injection to ensure that anesthesia was proceeding normally.

The animals were then randomly divided into the following groups (N=9):

| | GLuc-transfected | | | |
|---|---|---|---|---|
| Group | rUCMSCs^{A} | ALA | coelenterazine | DFO^{B} |
| 1 | + | + | + | + |
| 2 | + | + | + | − |
| 3 | + | + | − | + |
| 4 | + | + | − | − |
| 5 | + | − | − | − |

| | |
|---|---|
| 6 | Saline Only |
| 7 | No Tumor - Control |

^{A}Gaussia luciferase-transfected rat umbilical cord matrix-derived stem cells. ^{B}Deferoxamine.

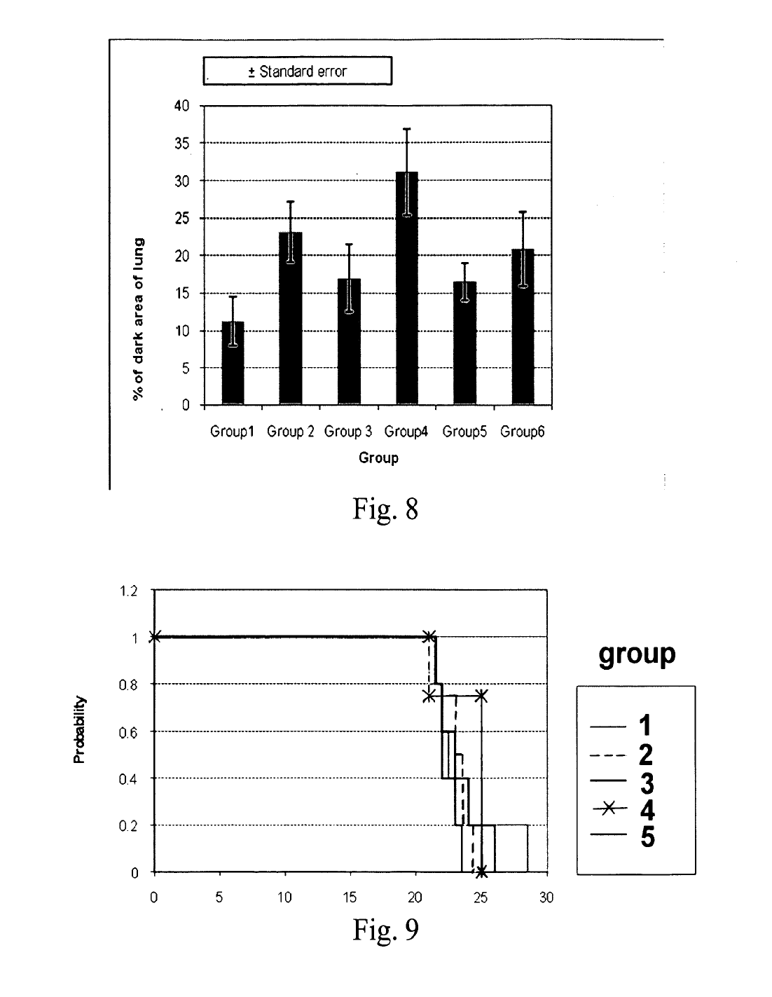

On Day 6, GLuc-transfected rUCMSCs were administered, as indicated, via an i.v. injection as 500,000 cells/mouse (200 μL volume). Twenty-four hours later, an iron chelator, DFO was then administered as 200 mg/kg in water (4 mg/mouse), intraperitoneal (i.p.) injection (100 μL). On Day 7, ALA was administered as 100 mg/kg in water (2 mg/mouse) via an i.p. injection (100 μL). On Day 8, the substrate coelenterazine was administered i.v. as 5 mg/kg in water (administered as β-cyclodextrin-complexed coelenterazine, prepared by dissolving 2 mg coelenterazine in 1 mL methanol with 100 mg cyclodextrin to form a complex, then dried, and re-dissolved in water) (100 μg/mouse). Treatment was repeated on Day 9 by again administering GLuc-transfected rUCMSCs, followed by DFO on Day 10, and then ALA on Day 11. Four hours later, coelenterazine was administered. This procedure was repeated on Days 12, 14, and 14, respectively. The animals were sacrificed on Day 21. The results are shown in FIG. 8. The % of dark (cancerous) area of the lung was calculated using software (imageJ) available from the NIH.

### Example 5

### PDT and Bioluminescent Imaging of Lung Melanoma Model in Mice

In this Example, C57BL6 mice were inoculated with B16F10luc2 melanoma cells (200,000/mice i.v.) to create lung melanomas (Day 0). The 30 mice were divided into 5 treatment groups as follows.

| Group | Photosensitizer |
|---|---|
| 1 | None |
| 2 | THPC-iRGD + RuiRGD |
| 3 | ALA/DFO + THPC-iRGD |
| 4 | ALA/DFO + Ru-iRGD |
| 5 | THPC-iRGD + Ru-iRGD + ALA/DFO |

On Day 4, NSCs were plated in a T75 flask (50,000 cells/cm²) in DMEM medium with 10% fetal bovine serum(FBS) and 5% horse serum. The NSCs were neural progenitor cells isolated from neonatal mouse cerebellum (gift from Ourednik lab, Iowa State University, Cell 69,1992,33-51). 30 million cells were required for the 30 mice. The cells were then incubated in a regular incubator (Temp 37° C., CO₂ 5% humidity 95%). The NSCs were then transfected with the GLuc gene according to the manufacturer's protocol. Specifically, after 24 hours, the NSCs were about 80% confluence and TurboFect™ (Fermentas) was used for transient transfection with GLuc. After 24 hours of transfection of NSC with GLuc, transfection efficiency was confirmed by coelenterazine (50 microliter of GLuc secreted medium and 10 microliter of coelenterazine (1 mg/mL)). The transfected cells were then washed with PBS, lifted with trypsin, and counted.

On Day 6, 1 million NSC/GLuc cells were injected i.v. into the tail vain of each mouse. Determination of initial B16F10 tumor sizes was also carried out via IVIS Imaging (the ventral side of mouse was saved and tumor size was measured using IVIS after 5 min. of i.p. injection of D-luciferin (150 mg/kg)). On Day 8, DFO was dissolved in distilled autoclaved water (DAW) (40 mg/mL) and injected i.p. at 200 mg/kg. Additional NSC plating for Glue transfection was carried out on Day 8 as well. On Day 9, the indicated photosensitizing agent was administered i.v. into the tail vein of each mouse. ALA was first dissolved in DAW (20 mg/mL) and injected i.v. 100 mg/kg. THPC-iRGD photosensitizing agent was dissolved in DAW (0.3 mg/kg; 0.060 μg/mL) and injected i.v., (100 μL). Ru-iRGD photosensitizing agent was dissolved in DAW (0.874 mg/Kg, 0.174 mg/mL) and injected i.v. (100 μL). Four hours later, coclenterazine was then mixed 1 mg in 50 mg of cyclodextrin (first dissolved in methanol, dried in vacuum and re-dissolved in DAW (51 mg/mL ˜1 mg coel.)) and then injected retroorbitally (“RO;” 100 μg). The NSCs plated on Day 8 were also transfected with Glue on Day 9. On Day 10, the transfected cells were injected i.v. into the tail vein of each mouse as described above. On Day 12, DFO was administered as described above. Additional NSCs were also plated for transfection. On Day 13, the photosensitizing agents were administered as described above. Treatment occurred four hours later, when cyclodextrin-complexed coelenterazine was injected RO as described above. For further treatments, the NSCs plated above were also transfected with GLuc on Day 13. On Day 14, the treatment process was repeated by injecting the additional transfected cells i.v. into the tail vein of each mouse. On Day 15, DFO was administered as described above, followed by the photosensitizing agents, and coelenterazine.

Mouse survival rates were then determined. The results are shown in FIG. 9. It was seen that the average tumor volume decreased in the PDT treated groups; however, the overall survival rate was not statistically different between the groups. The lung melanoma model used for these studies appears to be too aggressive for this length of a study.

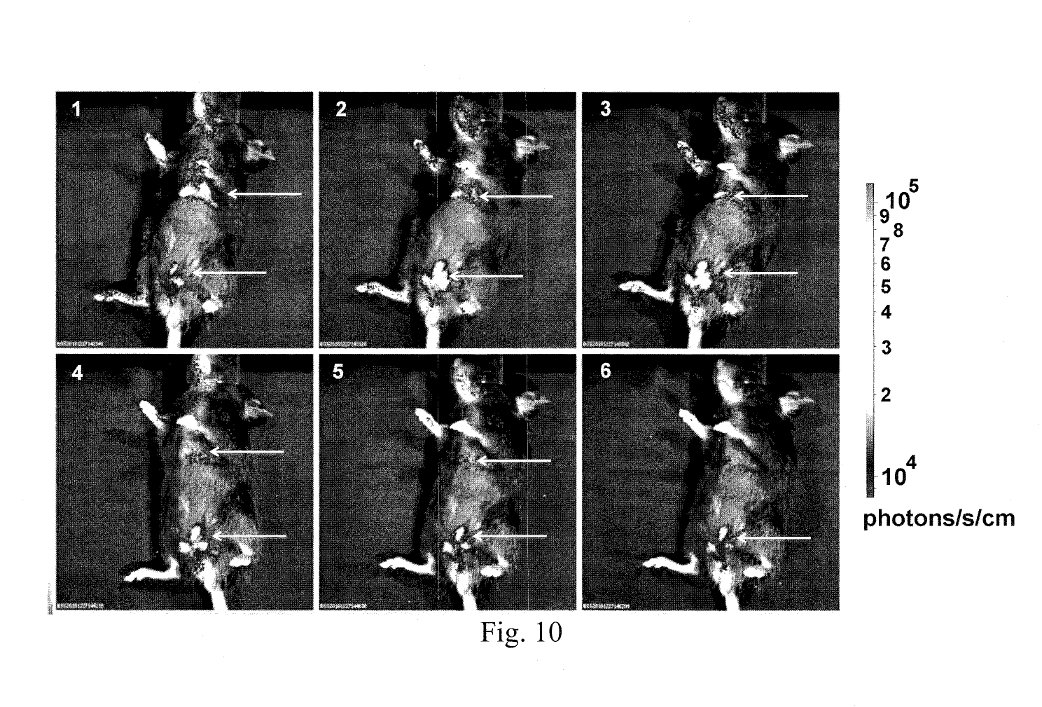

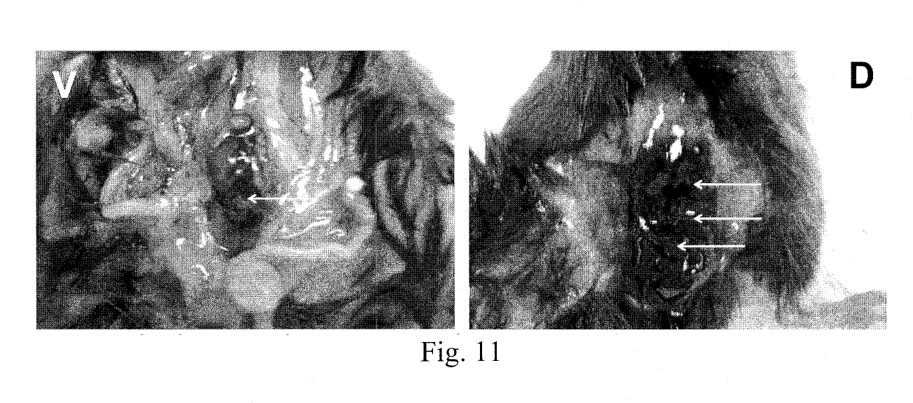

A surprising finding, however, was that additional tumors had formed in the mice in completely unexpected locations. When the mice were imaged using the IVIS equipment, the luminescence from these previously-unknown tumors could be seen. But for the imaging, the presence of these tumors may have remained undiscovered. The results of a typical mouse from Group 4 are shown in FIG. 10, taken directly after coelenterazine injection, and after 5-, 11-, 17-, 23-, and 28-minutes post-injection. The images were recorded using an IVIS 200 Imaging System 120v (includes XGI-8 Gas Anesthesia System and XWS-248 Workbench. The images show that the luminescence occurs from the ruthenium-complex (Ru-iRGD), and the emission occurs around 700 nm, not 480 nm (as this would be typical for the emission occurring from Gaussia luciferase in the presence of its substrate coelenterazine). Thus, although the PDT did not prolong the survival rates of the mice, the present study unexpectedly demonstrated superior imaging capabilities of this platform as well, indicating the usefulness of the invention for not just therapeutics, but also diagnostics. The presence of tumors at the unexpected locations was confirmed after euthanasia (FIG. 11).

It is remarkable that this imaging approach using tumor-homing neural stem cells that are transfected with the gene for the expression of Gaussia luciferase production will work for small tumors (early primary tumors and metastases). In this study, imaging resulted in identification of metastatic peritoneal tumors we had no idea were there. Based upon this data, it is estimated that tumors smaller than 1 mm in diameter can be imaged. Using ALA (that triggers the production of protoporphyrin IX in cancer cells), or any ruthenium-polypyridyl complex, porphyrin, chlorin, or bacteriochlorin, these tumors can be imaged. We are able to link any tumor- or cell-homing oligopeptide to the sensitizers (except ALA, where this is not necessary) in order to facilitate enhanced tumor uptake, resulting in very specific imaging capabilities.

### Example 6

### Deep-Tissue PDT Treatment of Metastatic 4T1 Breast Cancer in Mice

In this Example, PDT using in-situ generation of light was used to treat metastatic breast cancer in BALB C mice. ALA was used as the only photosensitizer/prodrug, and DFO was used as the iron chelator. The mice were divided into three treatment groups as follows.

| Group | # Mice (n) | Photosensitizer | DFO | NSC/Gluc | Coelenterazine |
|---|---|---|---|---|---|
| 1 | 5 | − | − | − | − |
| 2 | 5 | − | + | + | − |
| 3 | 6 | + | + | + | + |

On Day 0, the mice were inoculated with 4T1luc2 breast cancer cells (100,000/mouse, i.v.). On Day 3, 25 million NSCs were plated as described above for transfection. On Day 4, the cells were transfected with Glue. On Day 5, IVIS imaging with D-luciferin was carried out in the morning to determine tumor size and stratification of the mice. In the afternoon, the mice in Groups 2 and 3 were injected with the transfected cells (1 million/mouse). On Day 7, DFO was administered i.p. (300 mg/kg), as described above. On Day 8, the photosensitizing agent, ALA, was injected i.p. (100 mg/kg). Four hours later, cyclodextrin-complexed coelenterazine was administered i.v. (100 μg) into the tail vein of each mouse. The ALA/coelenterazine procedure was repeated on Day 9, and then on Day 10. The control groups were injected with PBS.

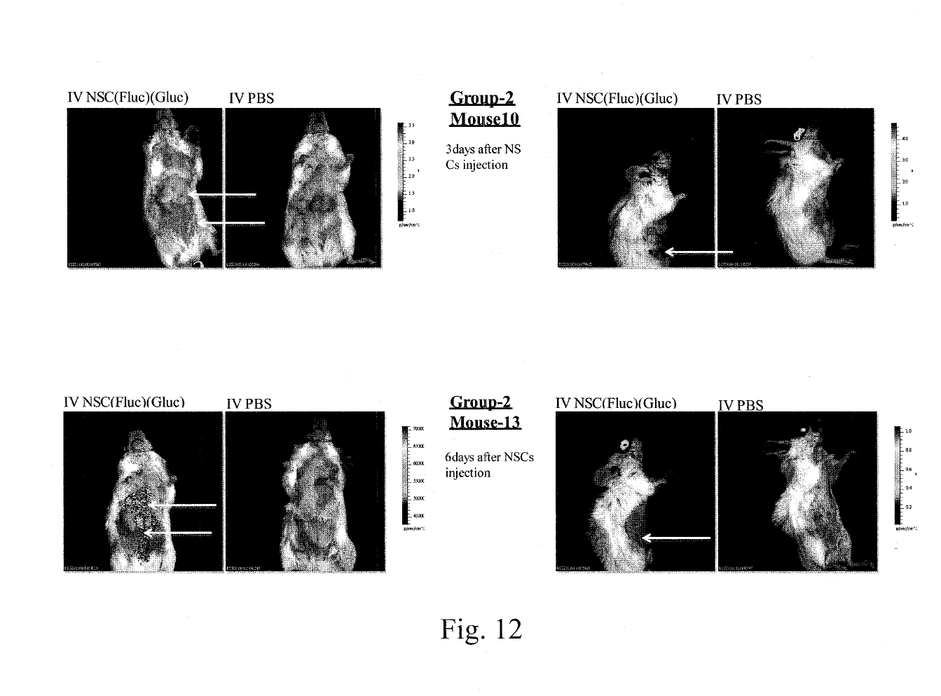

Images are shown in FIG. 12. It can be seen that the NSCs infiltrated the tumor tissue, expressed luciferase lighting up only the tumor tissue, allowing imaging of the cancerous tissue. The images were recorded using an IVIS 200 Imaging System 120v (includes XGI-8 Gas Anesthesia System and XWS-248 Workbench.

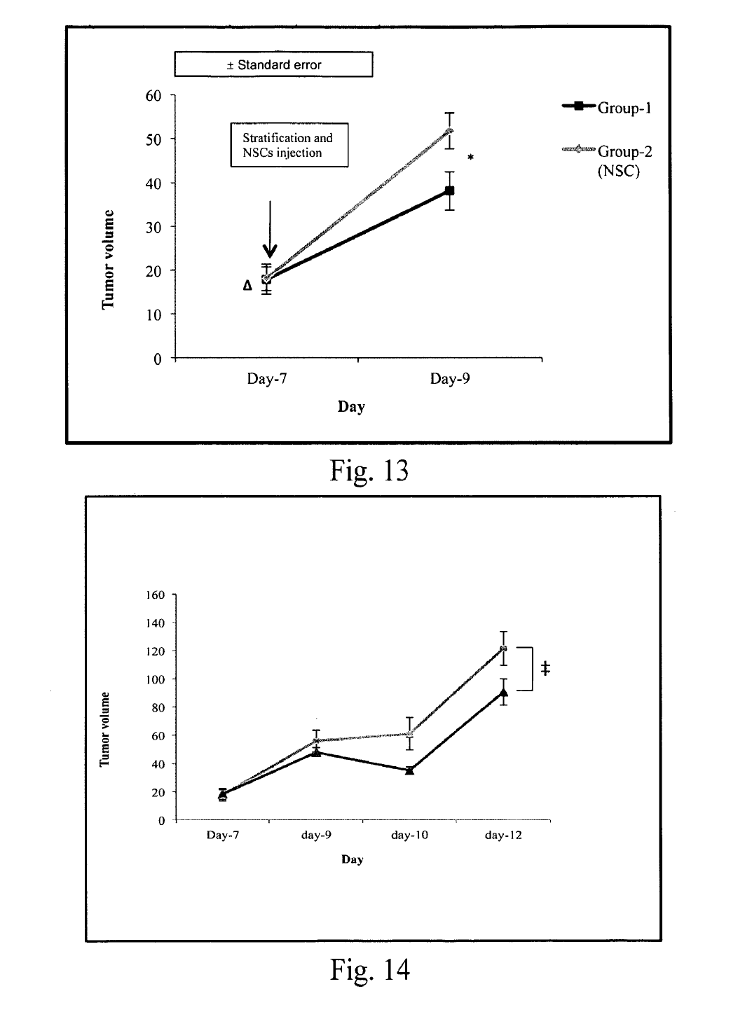

The tumor volume was found to increase significantly in group 2, compared to the control group 1 (FIG. 13). This finding is attributed to the migration of neural stem cells into the tumor, as there was a significant difference after NSC injection (significance fixed at 0.1 p-value). As shown in the results, a statistically significant difference (p-value<0.1) was observed for group 3, which received PDT treatment, as compared with to group 2, which only received the stem cell treatment and iron chelator, but not the photosensitizer/prodrug or substrate for PDT. The results are shown in FIG. 14. These results indicate that the photodynamic treatment of deep-seated tumors via stem-cell mediated photodynamic therapy was effective for decreasing tumor volume.

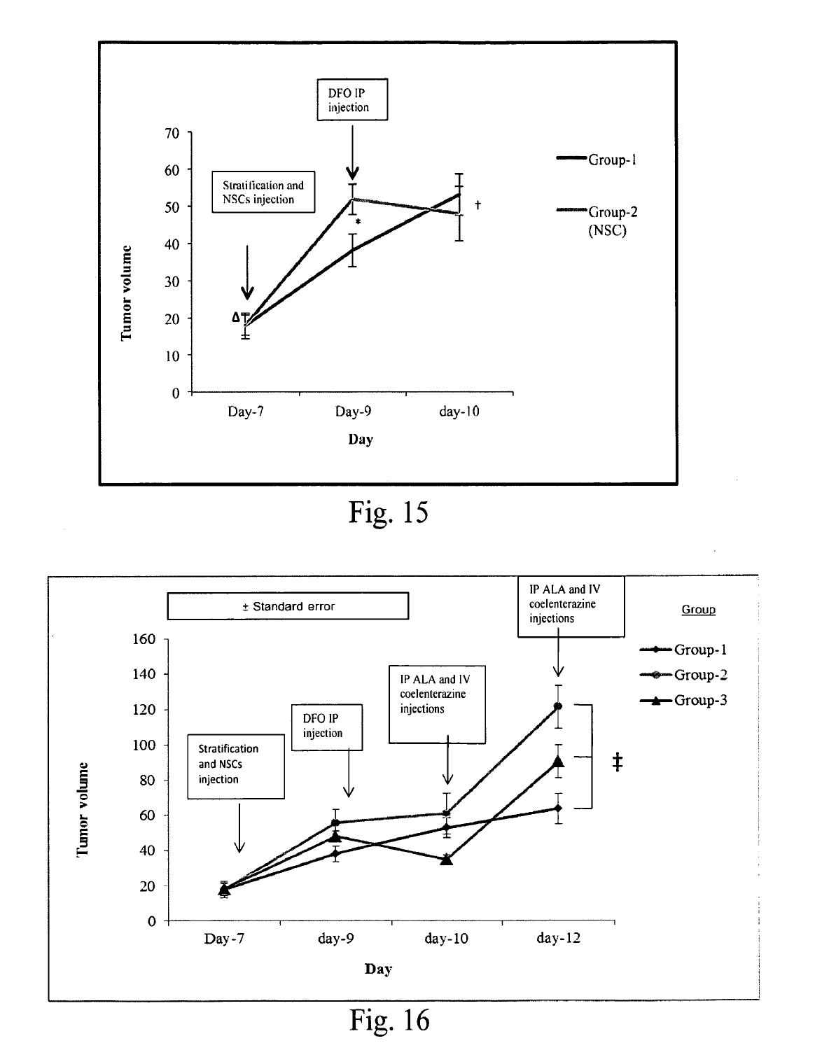

Interestingly, the iron chelator was found to have a mild effect in suppressing tumor growth (p-value: 0.65), although the size of each group was too small to observe a statistically more significant effect. Since the concentration of iron(II) is enriched in tumor tissue and the absence of iron(II) due to the chelator prohibits the biosynthesis of heme from the protoporphyrin 1× generated by the cancerous tissue, DFO may have a dual effect in PDT treatment. These results are shown in FIG. 15. A comparison of all groups is shown in FIG. 16.

### Example 7

### Synthesis of Coelenterazine

Coelenterazine (2-(4-hydroxybenzyl)-6-(4-hydroxyphenyl)-8-(phenylmethyl)imidazo[1,2-a]pyrazin-3(7H)-one), is a luminogenic compound found in different marine organisms including Aequorea victoria jellyfish, Renilla reniformis sea pansy and Watasenia scintillans squid. It forms blue fluorescence protein (BFP) in the presence of molecular oxygen in jellyfish which emits blue light with aid of calcium ion. Like other chemoluminescent compounds (luciferins), coelenterazine emits blue light (λmax=480 nm) in the presence of an enzyme (Renilla luciferase) outside the biological system without ATP. The molecular basis of the chemoluminescence of coelenterazine is shown in Scheme 2.1. Since no excitation light is required, there is no background fluorescence and, consequently, a much improved signal to noise ratio in imaging experiments. Furthermore, light of blue wavelength is better suited to excite potent photodynamic agents, such as porphyrins and ruthenium(II)polypyridyl-complexes. It must be noted that blue light from Renilla luminescence has a very low penetration in human tissue (1-2 mm), so that external irradiation in photodynamic treatment is very difficult. Therefore, our chemoluminescent system has a significant advantage, because it allows in-situ-photodynamic therapy.

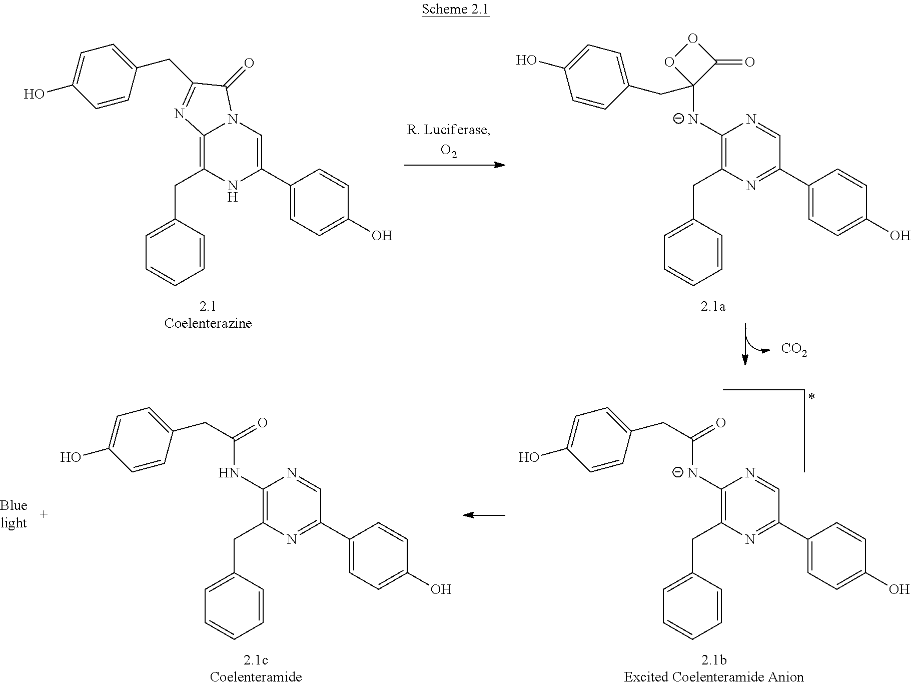 [SMILES: O=c1c(Cc2ccc(O)cc2)nc2c(Cc3ccccc3)[nH]c(-c3ccc(O)cc3)cn1-2 ; O=C1OOC1(Cc1ccc(O)cc1)[N-]c1ncc(-c2ccc(O)cc2)nc1Cc1ccccc1 ; "CO2" ; O=C(Cc1ccc(O)cc1)Nc1ncc(-c2ccc(O)cc2)nc1Cc1ccccc1 ; O=C(Cc1ccc(O)cc1)[N-]c1ncc(-c2ccc(O)cc2)nc1Cc1ccccc1]

The total synthesis of this bioluminescent compound was first reported by Inoue et al. in 1975. Since then many methods for the synthesis of coelenterazine have evolved. Although coelenterazine is commercially available, it is very expensive and not long-term stable. Therefore, the goal was to modify the existing methods for the synthesis of coelenterazine to provide it highly pure and in large quantities (gram scale) from inexpensive starting materials.

All chemicals were obtained from Acros Organics and Sigma Aldrich, unless noted otherwise. Tetrahydrofuran (THF) was distilled over sodium in presence of benzophenone in argon atmosphere. N,N-dimethyl acetamide (DMA) was distilled over calcium hydride in an argon atmosphere. All other solvents were used without distillation unless otherwise noted. 400 MHz and 200 MHz Varian NMR-spectrometers, Thermo Scientific Nicolet 380 FT-IR (neat sample, thanks to Prof. Dr Aakeröy, Department of Chemistry at KSU) were used in this study. We would like to thank the Analytical Laboratory of Dr. Ruth Welti at KSU for recording the mass spectra of our compound employing an Applied Biosystems API-4000 triple quadrupole mass spectrometer with electrospray and APCI sources.

### 1. Synthesis

### a. 3,5-dibromo-2-aminopyrazine (molecular formula C₄H₃Br₂N)

Synthetic procedure: 3,5-dibromo-2-aminopyrazine 2.3 was synthesized by modifying the bromination procedure of aminopyrazine. First, aminopyrazine 2.2 (1.00 g, 10.5 mmol, 1.0 eq) was completely dissolved in 75 mL dichloromethane (DCM) in an argon flushed 500 mL RB flask, and then N-bromosuccinimide (NBS) (4.683 g, 26.3 mmol, 2.5 eq) was added at once. The reaction mixture was stirred for 2 hours at room temperature (RT). The reaction was completed within 2 hours, which was confirmed by TLC. Reaction mixture was poured in 500 mL of 5% Na₂CO₃ and extracted with dichloromethane (3*100 mL) and washed with water (3*100 mL). The organic layer was dried using sodium sulfate and concentrated via rotary evaporation. Finally the dibromo-aminopyrazine was purified by descending silica gel column chromatography with 1:2 ethyl acetate:hexane as eluent. A pale yellow needle shaped crystalline compound was obtained 2.3 (1.555 g, 6.17 mmol, 62% yield). R_{f} value of the compound on SiO₂ thin layer chromatography (TLC) using 2:1 hexane:ethylacetate as mobile phase is 0.46. Melting point (MP) of the compound is 115-116° C. IR (neat) wavenumber (ν) cm⁻¹ 635, 877, 907, 1040, 1096, 1133, 1314, 1450, 1506, 1548, 1620, 3150, 3168, 3280, 3448; ¹H NMR (CDCl₃, 400 MHz) δ [ppm] 5.105 (b s, 211, NH₂) 8.04 (s, 1H, Ar—H); ¹³C NMR (CDCl₃, 400 M Hz) δ [ppm]123.87, 124.15, 143.38, 152.11; MS C₄H₃Br₂N3 m/z calculated 250. found 251.1 (M⁺).

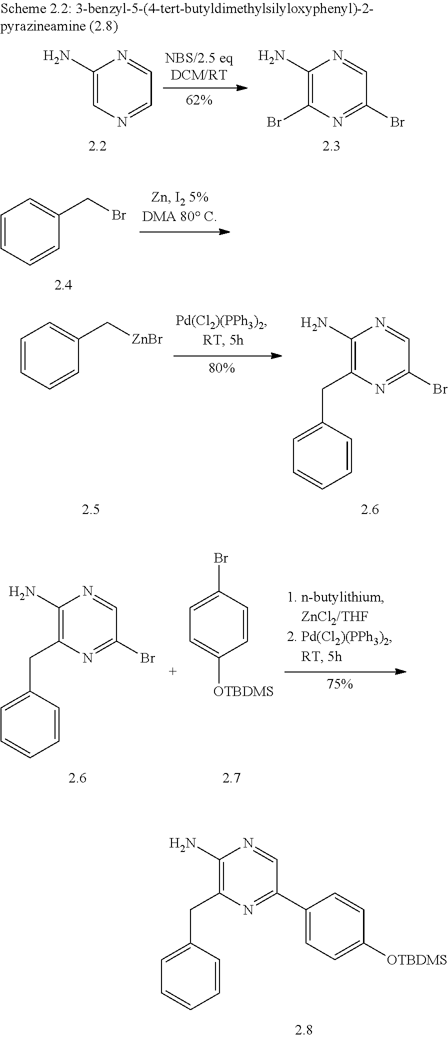

### b. 3-Benzyl-5-bromo-2-amino-pyrazine (2.6)

Synthetic procedure: The Huo modification of the Negishi coupling reaction was adapted for this reaction. Zn dust (1140 mg, 8.71 mmol, 3.5 eq.) and I₂ (216 mg, 5% of Zn) were added to a dry 25 mL two-necked round bottom flask under argon atmosphere. 5 mL of anhydrous dimethylacetamide (DMA), freshly distilled over calcium hydride, was added using a syringe. The mixture was stirred at RT until the brown color of I₂ disappeared. Then freshly distilled benzyl bromide (1526 mg, 4.46 mmol, 2.5 eq) was added by using a syringe and the reaction mixture was stirred at 80° C. for 3 hours. After insertion of Zn, the reaction mixture was cooled to RT and the suspension of dibromoaminopyrazine 2.3 (900 mg, 3.571 mmol, 1 eq.) and PdCl₂(PPh₃)₂ (126 mg, 0.178 mmol, 5% of pyrazine) in 6 mL of DMA was added. The reaction mixture was continuously stirred for 5.5 hours. Then the mixture was poured in 25 mL of water and extracted with ethyl acetate (3*25 mL). The organic fractions were combined and dried over anhydrous sodium sulfate and then concentrated on a rotary evaporator. Compound 2.6 (830 mg, 1.4 mmol, 88% yield) brown viscous oil was purified by silica gel descending column chromatography using 2/1, n-hexane/etylacetate (v/v) as an eluent. TLC (silica, mobile phase: n-hexane/ethyl acetate 2/1 v/v, R_{f}=0.35). IR (neat) wavenumber (ν) cm⁻¹633, 923, 1072, 1118, 1220, 1388, 1423, 1604, 2921, 3023, 3060, 3203, 3317, 3438; ¹H NMR (CDCl₃, 400 MHz) δ [ppm] 4.065 (s, 2H, CH₁) 4.449 (br s, 2H, NH₂), 7.198-7.317 (m, 5H, Ar—H), 8.012 (s, 1H, Ar—H); ¹³C NMR (CDCl₃, 200 MHz) δ [ppm] 41.11, 126.428, 127.503128.592, 129.304, 135.837, 141.909, 142.635, 152.253, MS C₁₁H₁₀BrN₃ m/z calculated 264. found 264 (M⁺), 265 (M+1).

### c. 3-Benzyl-5-(4-tert-butyldimethylsilyloxyphenyl)-2-pyrazineamine (2.8)

Synthetic procedure: This procedure is a modification of the Buchwald variant of a Negishi-type cross coupling reaction between aryl halides. A 25-ml round-bottom flask was dried overnight at 150° C., allowed to cool down to RT under argon atmosphere and then filled with (4-bromop-phenoxy)-tert-butyldimethylsilane 2.7 (1.984 g, 6.908 mmol, 2.2 eq) and 12 mL tetrahydrofuran (THF) (distilled over sodium in presence of benzophenone). A septum was used to block the influx of air. The mixture was cooled to −78° C., then n-butyl-lithium from a 2.8 molar solution in hexane (3.03 mL, 8.792 mmol, 2.8 eq) was added drop-wise by means of a syringe. The resulting solution was stirred at −78° C. for one hour. ZnCl₂ (1.283 mg, 9.42 mmol in 10 mL THF, 3 eq) was added via syringe through the septum. Again, the reaction mixture was stirred for 0.5 hours at −78° C. Then the reaction mixture was allowed to warm up to RT and the solution was further stirred for one additional hour at RT. A suspension of PdCl₂ (PPh₃)₂ (110 mg, 0.157 mmol, 5% mol) and pyrazine 2.6 (830 mg, 3.14 mmol, 1 eq) in 10 mL of THF was added and stirred at RT. The reaction progress was monitored by TLC (silica, mobile phase: n-hexane/ethyl acetate 2/1 v/v, R_{f} values for starting material, product and by-product(s) are 0.4, 0.29 and 0.55 respectively). There was some starting material discernible after five hours of reaction, but after 15 hours there was no more starting material. There was a minor fraction of by-product, which was identified as the product from the “unwanted coupling reaction” (R_{f}=0.29, silica, mobile phase: n-hexane/ethyl acetate 2/1 v/v), but the main fraction consisted of the product (R_{f}=0.55, silica, mobile phase: n-hexane/ethyl acetate 2/1 v/v). The reaction mixture was poured into 50 mL of water, extracted with ethyl acetate (3*50 mL) and dried over anhydrous sodium sulfate. After drying the organic fraction, ethyl acetate was removed by using a rotary evaporator. The product 2.8, yellow solid (897 mg, 2.294 mmol, 73%), was purified by descending silica gel column chromatography by using 2/1 hexane/ethylacetate. IR (neat) wavenumber (ν) cm⁻¹ 728, 834, 918, 1170, 1237, 1251, 1454, 1519, 1609, 2851, 2933, 3145, 3284, 3485; ¹H NMR (CDCl₃, 400 MHz) δ [ppm]0.22 (s, 6H, 2CH₃), 1.005 (s, 9H, tert-butyl), 4.17 (s, 2H, CH₂), 4.37 (br s 2H, NH₂), 6.92 (d, J=8.59 Hz 2H), 7.25-7.32 (m, 5H), 7.81, (d, J=8.59 Hz, 2H), 8.32 (s, 1H, Ar—H); ¹³C NMR (CDCl₃, 400 MHz) δ [ppm] 4.1, 18.48, 25.9, 41.4, 120.7, 127.1, 127.2, 128.7, 129.1, 130.8, 137, 137.1, 140.6, 142.9, 151.6, 156.2, MS C₂₃H₂₉N₃OSi m/z calculated 291.58. found 391.8 (M⁺), 392.8 (M+1).

### d. (4-bromo-phenoxy)-tert-butyl-dimethyl-silane (2.7)

Synthetic procedure: 4-bromophenol 2.9 (2 g, 11.56 mmol, 1 eq) and 4-DMAP (0.141 g, 1.156 mmol) were dissolved in 30 mL DCM in a 100-ml round-bottom flask at 0° C. After 15 min imidazole (1.18 g 17.34 mmol, 1.5 eq) and TBDMS-C1 (2.613 g, 17.34 mmol, and 1.5 eq) were added and stirred at 0° C. for one additional hour. DCM was removed by using a rotary evaporator and the resulting solid was dissolved in 25 mL of diethyl ether. The diethyl ether solution was first washed with concentrated ammonium solution (2*50 mL), followed by water (2*50 mL), and finally brine solution (2*50 mL). Diethyl ether was removed via rotary evaporation to leave compound 2.7 behind, which was obtained as a colorless liquid (3.293 g, 11.46 mmol, 99% yield). ¹H NMR (CDCl₃, 400 MHz) 8 [ppm]0.16 (s, 6H), 0.95 (s, 9H), 6.69 (d, J=88.78, 2H), 7.29 (d, J=8.78 Hz, 2H); ¹³C NMR (CDCl₃, 400 MHz) δ [ppm] 4.2618.42, 25.85, 113.83, 122.13, 132.51, 155.07.

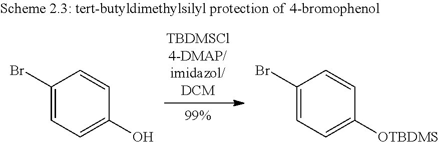 [SMILES: Oc1ccc(Br)cc1 ; CC(C)(C)[Si](C)(C)Oc1ccc(Br)cc1]

### e. 4-benzyloxybenzylalcohol (2.12)

Synthetic procedure: 2.0 g (16.11 mmol) of 4-hydroxybenzyl alcohol 2.10 and 11.132 g (80.5 mmol, 5 eq) of anhydrous potassium carbonate were dissolved in 40 mL dry DMF in a 100-ml round-bottom flask. The suspension of the mixture was purged with argon and stirred for 30 minutes at room temperature. Then 6.886 g (40.02 mmol, 2.5 eq) of benzyl bromide was added by means of a syringe to the suspension and stirred for 24 hours at RT. Then reaction mixture was filtered through celite and the celite cake was washed with 50 mL of diethyl ether. The filtered solution was washed with water (3*30 mL), followed by concentrated brine solution (2*25 mL). The diethyl ether solution was dried over anhydrous sodium sulfate and the solvent was evaporated by using a rotary evaporator and then further in a high vacuum. This experimental procedure yielded a white solid 3.12 g (15.58 mmol, 96% yield) of pure 4-benzyloxybenzylalcohol 2.12. R_{f} value of the compound is 0.75, 1/1 ethyl acetate/n-hexane, v/v as mobile phase in pre coated SiO, thin layer chromatography (TLC). MP 73-75° C. IR (neat) wavenumber cm⁻¹ 612, 694, 739, 810, 994, 1237, 1380, 1509, 1585, 1605, 1723, 1171, 2864, 2913, 3051, 3060, 3321 ¹H NMR (CDCl₃, 400 MHz) δ [ppm] 4.63 (d, J=5.86 Hz, 2H), 5.08 (s, 2H), 6.98 (d, J=8.7 Hz, 2H), 7.30 (d, J=8.7 Hz, 2H), 7.43-7.37 (m 5H); ¹³C NMR (CDCl₃, 400 MHz) δ [ppm]65.28, 70.28, 115.20, 127.67, 128.20, 128.89, 129.43, 133.61, 137.17, 158.65.

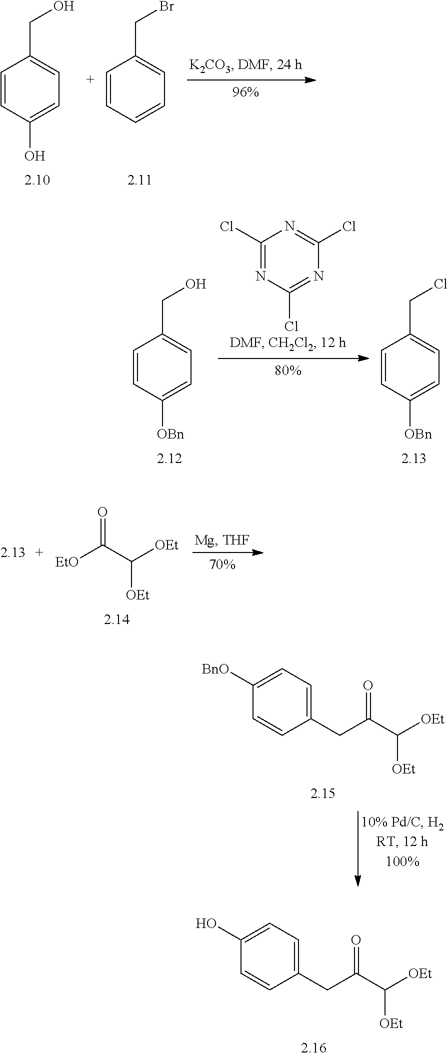

### f. 4-Benzyloxybenzylchloride (2.13)

Synthetic procedure: Surprisingly, the chlorination of benzyl alcohol turned out to be more difficult than initially expected. The chlorination of benzyl alcohol 2.12 was efficiently carried out by employing the cyanuric chloride(2,4,6-trichloro[1,3,5]triazine) and N,N-dimethyl formamide complexation method (CTC/DMF). Next, 0.369 g (1.997 mmol) of cyanuric chloride was dissolved in 1.0 mL of dry DMF in a two-necked 50 mL round-bottom flask. The solution was stirred at room temperature under argon atmosphere. After 30 minutes of stirring at RT, the white solid of the cyanuric chloride/DMF complex was formed. Then 0.40 g (1.997 mmol) of 2.12 (4-benzyloxybenzylalcohol) solution in 10 mL dichloromethane was added at once by means of a syringe to the white solid. The mixture was then stirred at room temperature. Reaction progress was monitored by TLC (silica, mobile phase: n-hexane/ethyl acetate, 10/1 v/v, R_{f} values for starting material, product are 0.09 and 0.63 respectively). The chlorination of benzyl alcohol was completed after 4 hours. After the completion of the reaction the reaction mixture was diluted to 20 mL by DCM. The white turbid suspension was filtered through celite, followed by washing the celite cake with 20 mL DCM. The solvent of the filtered solution was removed via rotary evaporation. This procedure yielded a white solid. The white solid was subjected to descending SiO₂ column chromatography by using 10% ethylacetate in hexane and yielded a fluffy white solid 2.13 0.31 g (1.417 mmol, 70.95%) MP 74-76° C. IR (neat) wavenumber (ν) cm⁻¹ 609, 660, 741, 834, 1004, 1171, 1241, 1380, 1512, 1580, 1609, 2868, 696, 2933, 3027; ¹H NMR (CDCl₃ 400 MHz) δ [ppm] 4.57 (s, 2H) 5.08 (s, 2H), 6.96 (d, J=8.7 Hz, 2H), 7.32 (d, J=8.7 Hz, 2H), 7.43-7.37 (m 5H); ³C NMR (CDCl₃, 400 M Hz) δ [ppm] 46.48, 70.28, 115.29, 127.67, 128.27, 128.84, 130.30, 136.97, 159.108.

### g. 2-Propanone, 1,1-diethoxy-3-(4-benzyloxyphenyl) (2.15)

Synthetic procedure: This method is modified form of Adamczyks method. 236 mg (10.289 mmol, 2.5 eq.) of magnesium were suspended in 3 mL of freshly distilled tetrahydrofuran (THF) in an argon flushed 50 mL two-necked round-bottom flask. Dibromoethane (0.10 mL) was added to activate the magnesium. After 20 minutes of activation, a solution of 0.90 g (4.115 mmol) of 2.13 in THF was added and stirred at RT for 30 minutes. The reaction mixture was further refluxed for one hour to complete the reaction. The pale yellow Grignard reagent was allowed to cool to RT and then kept on an ice bath. Then 906 mg (5.143 mmol, 1.25 eq) of ethyldiethoxyacetate 2.14 was dissolved in 10 mL THF in a separate 50 mL round-bottom flask under argon atmosphere and cooled to −78° C. The Grignard reagent was transferred drop-wise into the cooled flask during 10 minutes. The reaction mixture was then stirred for 1.5 hours at −78° C. Then reaction was quenched by 50 mL water and further diluted by 100 mL of ethylacetate. The ethylacetate layer was washed with water (2*50 mL), followed by saturated brine solution (2*50 mL). The organic layer was dried over anhydrous sodium sulfate and the solvent was evaporated by rotary evaporation. The viscous Grignard product was further purified by descending SiO₂ column chromatography 10% ethyl acetate in n-hexene (v/v) as an eluent. This procedure yielded 0.95 g (2.49 mmol, 70% yield) of the oily colorless compound 2.15. R_{f=0.4} (TLC, silica, mobile phase: n-hexane/ethyl acetate 10/1 v/v) ¹H NMR (CDCl₃, 400 MHz) δ [ppm]1.25 (t, J=7.3 Hz, 6H) 3.57 (m, 2H), 3.69 (m, 2H), 3.84 (s, 2H), 4.64 (s, 1H), 5.05 (s, 2H), 6.94 (d, J=8.4 Hz, 2H), 7.14 (d, J=8.4 Hz, 2H), 7.43-7.35 (m 5H); ¹³C NMR (CDCl₃, 400 MHz) δ [ppm]15.35 42.99, 63.53, 70.17, 115.5, 126.17, 127.66, 128.12, 128.76, 130.96, 132.007, 137.21, 157.94, 203.73.

### h. 2-Propanone, 1,1-diethoxy-3-(4-hydroxyphenyl) (2.16)

Synthetic procedure: The deprotection of the benzyl group of compound 2.15 was carried out by reduction using hydrogen on a palladium/carbon catalyst. 0.90 g (mmol 4.115) of compound 2.15 was dissolved in 50 mL of methanol in a 100-mL round-bottom flask. 100 mg of 10% palladium on carbon was added. At first the resulting suspension was put under vacuum and then hydrogen gas was passed into the RB flask from a balloon of hydrogen gas. The reaction mixture was stirred for 24 hours under hydrogen atmosphere. TLC (silica, mobile phase: n-hexane/ethyl acetate 1/10 v/v, R_{f} values for starting material, product were 0.4 and 0.16 respectively) was used to confirm the completeness of the deprotection of the benzyl group. The black suspension was filtered and the solvent was removed using a rotary evaporator. The deprotected product 2.16 was further purified by SiO₂ column chromatography using 50% ethylacetate in hexane, yielding 0.570 mg (2.392 mmol, 58%) colorless oil 2.16. ¹H NMR (CDCl₃, 400 MHz) δ [ppm]1.25 (t, J=7.03 Hz, 6H) 3.55 (m, 2H), 3.71 (m, 2H), 3.82 (s, 2H), 4.64 (s, 1H), 5.11 (br s, 1H), 6.77 (d, J=8.59 Hz, 2H), 7.07 (d, J=8.59 Hz, 2H); ¹³C NMR (CDCl₃, 400 MHz) δ [ppm]15.36, 43.08, 63.61, 102.42, 115.65, 125.86, 131.14, 154.83, 204.06, MS, m/z C₁₃H₁₈O₄ calculated 238.28. found 261.3 (M+Na).

### i. Coelenterazine (2.1

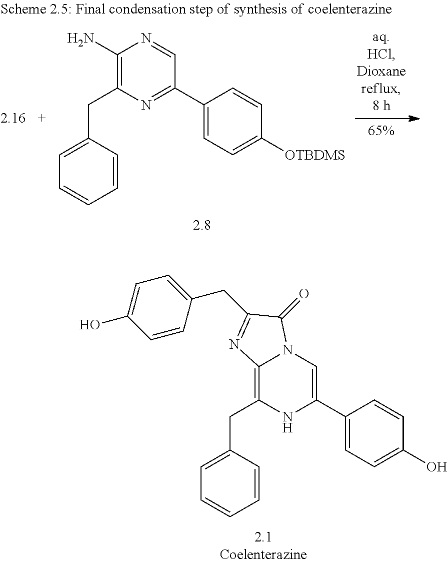 [SMILES: CC(C)(C)[Si](C)(C)Oc1ccc(-c2cnc(N)c(Cc3ccccc3)n2)cc1 ; O=c1c(Cc2ccc(O)cc2)nc2c(Cc3ccccc3)[nH]c(-c3ccc(O)cc3)cn1-2]

Synthetic procedure: The final condensation step was adapted from a published method. 200 mg (0.839 mg) of compound 2.16 was dissolved in 4.0 mL of degassed 1,4-dioxane (N₂, Ar, freeze-pump-and thaw) in a 50 mL two-necked flask. Then 0.60 mL of cone. HCl (38%) was dissolved in 2.0 mL of dioxane and added into the flask. Finally, a solution of 2.8 (328 mg 0.891 mmol) in 4.0 mL of dioxane was added into the mixture, which was then refluxed for 8 hours under argon atmosphere. After 8 hours of reflux, a dark brown solution was obtained, which was allowed to cool down to room temperature. The solvent was then removed by rotary evaporation. The resulting dark brown solid was subjected to descending SiO₂ column chromatography using 10% methanol in dichloromethane as eluent, yielding coelenterazine (0.543 mmol, 65%). R_{f}=0.42 (TLC silica, mobile phase: dichloromethane/methanol, 10/1, v/v), IR (neat) wavenumber cm⁻¹ 631, 695, 834, 1031, 1099, 1170, 1233, 1339, 1368, 1451, 1508, 1552, 1610, 2851, 2917, 3056, 3170; ¹H NMR (CD₃OD-d₄) δ [ppm] 4.06 (s, 2H) 4.39 (s, 2H), 6.68 (d, J=8.58 Hz, 2H), 6.86 (d, J=8.59 Hz, 2H), 7.14 (d, J=8.39 Hz, 2H), 7.18-7.22 (m, 2H), 7.29-7.25 (m, 2H), 7.38 (d, J=7.41 Hz, 2H), 7.48 (d, J=8.78 Hz, 2H), 7.62 (s, 1H); MS: m/z C₂₆H₁₂N₃O₃ calculated 423.15. found 424.3 (M+H), 446.3 (M+Na).

1. A method for photodynamic therapy of cancerous tissue comprising:
administering to a subject a therapeutically effective amount of tumor-trophic cells comprising a nucleic acid encoding for a luminescent protein;
administering a photosensitizing agent to said subject, wherein said photosensitizing agent is administered separately from said cells;
optionally administering an iron chelator to said subject; and
administering a luminogenic substrate corresponding to said luminescent protein to said subject;
wherein said substrate reacts with said luminescent protein to produce light, said light activating the photosensitizing agent which results in the damage and destruction of the cancerous tissue.

2. The method of claim 1, wherein said cells are selected from the group consisting of mammalian stem cells, monocytes, neutrophils, and combinations thereof.

3. (canceled)

4. The method of claim 1, wherein said cells are administered via intravenous injection, intraperitoneal injection, intramuscular injection, intratumoral injection, intraarterial injection, inhalation, or a combination thereof.

5. The method of claim 1, wherein about 500,000 to about 200 million cells are administered to said subject.

6. The method of claim 1, wherein said cells secrete said luminescent protein.

7. The method of claim 1, wherein said cells comprise a plasmid or vector comprising a nucleic acid encoding said luminescent protein.

8. The method of claim 1, wherein said luminescent protein is luciferase.

9. The method of claim 8, wherein said luminescent protein is selected from the group consisting of Renilla luciferase, Gaussia luciferase, firefly luciferase, and combinations thereof.

10. The method of claim 1, said cells having an emission spectrum, wherein said photosensitizing agent is selected so that the absorption spectrum of the photosensitizer is tuned to the emission spectrum of the cells.

11. (canceled)

12. The method of claim 1, wherein said photosensitizing agent is selected from the group consisting of free or metalated porphyrins, chlorins, benzoporphyrins, phthalocyanins, bacteriochlorins, cyanines, ruthenium complexes, photosensitizer-generating prodrugs, and combinations thereof.

13. The method of claim 1, wherein said photosensitizing agent is selected from the group consisting of tetrakis-4-carboxyphenyl-porphyrin, tris(N,N′″-bis(2-carboxyethyl)-4,4′:2,2″:4″,4′″-quaterpyridine-N′N″-dium-N′,N′″) ruthenium(II)octachloride, tetra-carboxyphenyl-chlorin sodium salt, 5 tetra-carboxyphenyl-bacteriochlorin sodium salt, meso-tetra-hydroxophenyl-chlorin, aminolevulinic acid, porfimer sodium, and combinations thereof.

14. The method of claim 1, wherein said photosensitizing agent comprises a tumor-homing peptide sequence.

15. The method of claim 14, wherein said tumor-homing peptide sequence is selected from the group consisting of SEQ ID NOs. 1-25.

16. (canceled)

17. The method of claim 1, wherein said photosensitizing agent accumulates in said cancerous tissue.

18. The method of claim 1, wherein said iron chelator is selected from the group consisting of deferoxamine, dipyridylthiosemicarbazone-chelators, and combinations thereof.

19. The method of claim 1, wherein said substrate is selected from the group consisting of coelenterazine, luciferin, and combinations thereof.

20. (canceled)

21. (canceled)

22. A method of imaging cancerous tissue comprising:
administering tumor-trophic cells to a subject, said cells comprising a nucleic acid encoding for a luminescent protein;
administering a photosensitizing agent to said subject, wherein said photosensitizing agent is administered separately from said cells, and wherein said cells and said photosensitizing agent accumulate in and near said cancerous tissue;
optionally administering an iron chelator to said subject;
administering a luminogenic substrate corresponding to said luminescent protein to said subject, wherein said substrate reacts with said luminescent protein to produce light of a first wavelength, said light activating the photosensitizing agent, which emits light of a second wavelength; and
detecting said light of a second wavelength emitted from said photosensitizing agent to determine the location of the cancerous tissue in said subject.

23. A kit for the photodynamic therapy or imaging of cancerous tissue, said kit comprising:
a photosensitizing agent comprising a tumor-homing peptide attached thereto;
an optional iron chelator;
a luminogenic substrate; and
instructions for the administration thereof.

24. The kit of claim 23, further comprising instructions for selecting and transfecting tumor-trophic cells to deliver a luminescent protein to said cancerous tissue as the light source for said photodynamic therapy or imaging.

25. (canceled)

26. (canceled)

27. A method for photodynamic therapy of cancerous tissue, said method comprising:
(a) administering to a subject a therapeutically effective amount of tumor-trophic cells comprising a nucleic acid encoding for a luminescent protein;
(b) administering a photosensitizing agent to said subject, wherein said photosensitizing agent is administered separately from said cells;
(c) optionally administering a iron chelator to said subject;
(d) administering a luminogenic substrate corresponding to said luminescent protein to said subject, wherein said substrate reacts with said luminescent protein to produce light, said light activating the photosensitizing agent which results in the damage and destruction of the cancerous tissue; and
(e) repeating steps (a)-(d).

28. (canceled)
